(19) United States
(12) Patent Application Publication
(10) Pub. No.: US 2024/0316043 A1
(43) Pub. Date: Sep. 26, 2024

(54) BROMODOMAIN INHIBITORS FOR CANCER THERAPY

(71) Applicant: University of Tennessee Research Foundation, Memphis, TN (US)

(72) Inventors: Duane Miller, Collierville, TN (US); Lawrence Pfeffer, Memphis, TN (US); Yali He, Memphis, TN (US); Yinan Wang, Memphis, TN (US); Chuan Yang, Memphis, TN (US)

(73) Assignee: University of Tennessee Research Foundation, Memphis, TN (US)

(21) Appl. No.: 18/612,647

(22) Filed: Mar. 21, 2024

Related U.S. Application Data
(60) Provisional application No. 63/491,474, filed on Mar. 21, 2023.

(51) Int. Cl. A61K 31/4995; A61K 31/495; A61P 35/00; C07D 487/08

(52) U.S. Cl. CPC A61K 31/4995; A61K 31/495; A61P 35/00; C07D 487/08

(57) ABSTRACT

The present disclosure related to compounds of Formula (I):

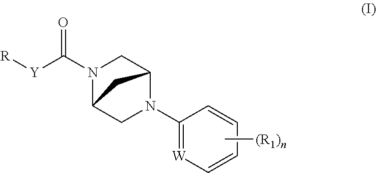

stereoisomers thereof, prodrugs thereof, and pharmaceutically acceptable salts thereof. The present disclosure also relates to uses of the compounds, including to treat cancer in a subject.

### CROSS REFERENCE TO RELATED APPLICATIONS

This application claims the benefit of U.S. Provisional Application No. 63/491,474, filed Mar. 21, 2023, which is hereby incorporated herein by reference in its entirety.

### TECHNICAL FIELD

The presently disclosed subject matter relates to bromodomain inhibitors, including novel selective inhibitors of the Brahma-related gene-1 (BRG1) bromodomain and to methods of using bromodomain inhibitors to treat cancer, particularly therapy-resistant glioblastoma. For example, in some embodiments, the presently disclosed subject matter relates to methods of treating glioblastoma comprising administering a BRI and a therapeutic agent, such as a DNA alkylating agent and/or radiotherapy, to a subject in need of treatment for glioblastoma.

### BACKGROUND

Glioblastoma (GBM) is the most common malignant primary brain tumor in adults and a leading cause of cancer-related morbidity and mortality in the United States.^{1, 2} Surgical resection of GBM, combined with adjuvant temozolomide (TMZ) chemotherapy and radiation therapy, remains the primary treatment modality, but its 5-year overall prognosis is dismal (<10% survival) which has remained unchanged for decades.¹ The aggressive and diffuse infiltrative nature of GBM makes it extremely difficult to treat. Furthermore, the rapid tumor recurrence after surgical resection and treatment by radiotherapy and chemotherapy demonstrates the ability of the residual GBM cells to evade treatment and propagate.

Bromodomains (BRDs) are a family of evolutionarily conserved motifs identified for the first time in the early 1990s in the brahma gene of Drosophila melanogaster.³ BRDs are compact protein modules comprising approximately 110 amino acids, which bind to acetylated lysine residues, such as those on the N-terminal tails of histones, and recruit other chromatin factors, thereby regulating gene transcription.⁴ There are a total of 61 highly diverse BRDs that have been identified in 46 different BRD-containing proteins encoded by the human genome.⁵ Of the 46 known, 11 had two bromodomains, and one protein had 6 bromodomains.⁶ They are classified into eight subfamilies according to its sequence homology. BRDs are increasingly being considered as attractive therapeutic targets for a variety of diseases including inflammatory and cardiovascular diseases, and cancer. BRDs play the critical role in control of target genes that are difficult to modulate directly with small molecules.

BRG1 and BRM contain BRDs and are the catalytic subunits of the SNF/SWI chromatin-remodeling complex. BRG1 and BRM plays key roles in regulating gene expression and transcription control by disrupting histone-DNA contacts in an ATP-dependent manner. Loss of components of SWI/SNF has been linked to cancer development.⁷ BRG1/BRM BRD targeting chemical probes would be useful tools to understand the modulation of SWI/SNF mediated processes, and any inhibitors identified could also offer starting points for druglike lead compounds of potential therapeutic utility in cancers and other diseases. PFI-3 was identified as a broadly selective, potent and cellular active inhibitor of family VIII BRDs including BRG1/BRM/PB1.⁸ PFI-3 showed excellent binding affinities for PB1 (Kd 54 nM), and BRG1/BRM (Kd<0.1 μM) and excellent selectivity for these sub-family VIII BRDs in a panel against >40 BRDs from other families.^{9, 10} Recently published are the structural-related analogs of PFI-3 that target the BRG1 catalytic subunit of the SWI/SNF complex.¹¹ Although inhibitory effects were not observed against a range of cellular endpoints in 12 primary human cell based systems when incubated with PFI-3 and its analogs,⁹ it was found that PFI-3 and its analogs enhanced the antiproliferation activity and cell death-inducing effects of temozolomide (TMZ) in both TMZ-sensitive GBM cells and TMZ-resistant GBM cells.^{11, 12} In an intracranial GBM animal model, it was also found that PFI-3 and its analogs showed an increase in survival of animals bearing GBM tumors that were treated with TMZ.^{11, 12}

Whereas BRG1 and BRM are strongly linked to cancer development and there is a synthetic lethal relationship between BRG1 and BRM, developing novel selective BRG1 and/or BRM BRD inhibitors is needed to explore whether the BRD of BRG1 and/or BRM represents a druggable target in brain tumors. Inhibiting BRG1 by genetic or pharmacologic means promotes glioma cell differentiation, loss of cancer stemness and sensitizes glioma to the effects of alkylating agents, such as TMZ and carmustine, which are standard treatment for GBM patients.^{2, 11, 13, 14} Taken together, the BRG1 and/or BRM subunits of SWI/SNF have been identified as novel druggable therapeutical targets in GBM and revealed the therapeutic potential of applying novel small molecule BRG1 inhibitors of SWI/SNF to improve the standard-of-care treatment for GBM.

Accordingly, there is an ongoing need for identifying new therapies and molecular targets in the treatment of GBM, particularly for the development of new therapeutic strategies for targeting therapy-resistant cells in GBM.

### SUMMARY

In some embodiments, the presently disclosed subject matter provides a compound having the structure of Formula (I):

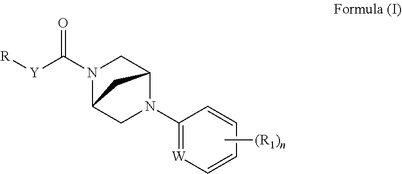

Wherein:
R represents alkyl, cycloalkyl, alkenyl, cycloalkyl, alkynyl, phenyl, aryl, heteroaryl or arylalkyl moiety having at most 12 carbon atoms, these moieties being optionally substituted by one or more substituents chosen from the following functional groups: optionally esterified, etherified or protected hydroxy, alkoxy, hydroxyalkyl, alkenyloxy, alkynyloxy, trifluoromethyl, mercapto, halogens, cyano, nitro, acyl, acyloxy, aryl, esterified, amidified or salified carboxy, amino, mono- or dialkylamino, or hydrogen.
R₁=alkyl, alkoxy, hydroxy, esterified, etherified or protected hydroxy, amino, mono- or dialkylamino, halogens (F, Cl, Br, I), acyl, acyloxy, cyano, trifluoromethyl, methoxy, nitro, esterified, amidified or salified carboxy, and hydrogen, etc.;
Y═O, NH, CH₂, bond;
W═CH, N;
n is an integer from 0 to 4;

In some embodiments, the compound of Formula (I) is selected from:

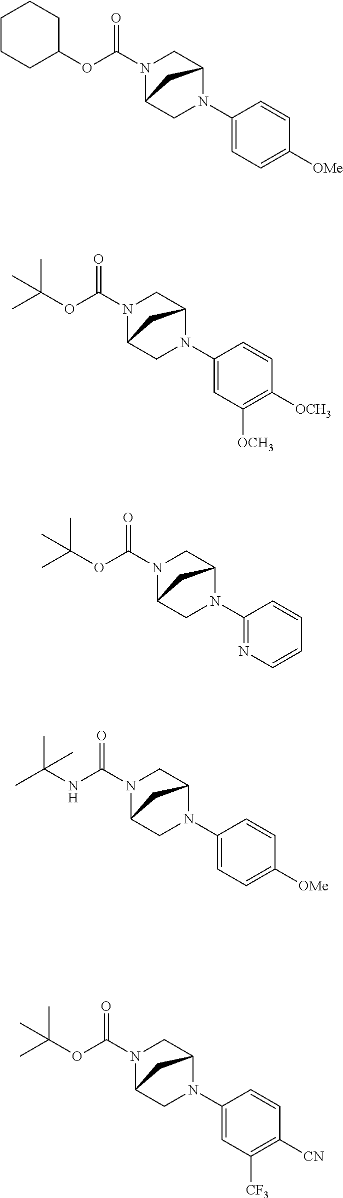 [SMILES: COc1ccc(N2C[C@H]3C[C@@H]2CN3C(=O)OC2CCCCC2)cc1 ; COc1ccc(N2C[C@H]3C[C@@H]2CN3C(=O)OC(C)(C)C)cc1OC ; CC(C)(C)OC(=O)N1C[C@H]2C[C@@H]1CN2c1ccccn1 ; COc1ccc(N2C[C@H]3C[C@@H]2CN3C(=O)NC(C)(C)C)cc1 ; CC(C)(C)OC(=O)N1C[C@H]2C[C@@H]1CN2c1ccc(C#N)c(C(F)(F)F)c1]

or a pharmaceutically acceptable salt thereof.

In some embodiments, the compound of Formula (I) has a structure of Formula (II):

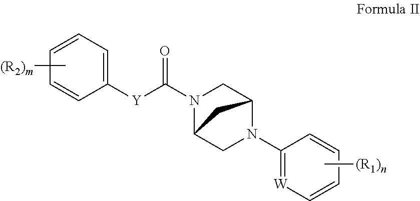

Wherein:
R₁=alkyl, alkoxy, hydroxy, esterified, etherified or protected hydroxy, amino, mono- or dialkylamino, halogens (F, Cl, Br, I), acyl, acyloxy, cyano, trifluoromethyl, methoxy, nitro, esterified, amidified or salified carboxy, and hydrogen, etc.;
R₂=alkyl, alkoxy, hydroxy, esterified, etherified or protected hydroxy, amino, mono- or dialkylamino, halogens (F, Cl, Br, I), acyl, acyloxy, cyano, trifluoromethyl, methoxy, nitro, esterified, amidified or salified carboxy, and hydrogen, etc.;
Y═O, NH, CH₂, bond;
W═CH, N;
m or n is an integer from 0 to 4;

In some embodiments, the compound of Formula (II) is selected from:

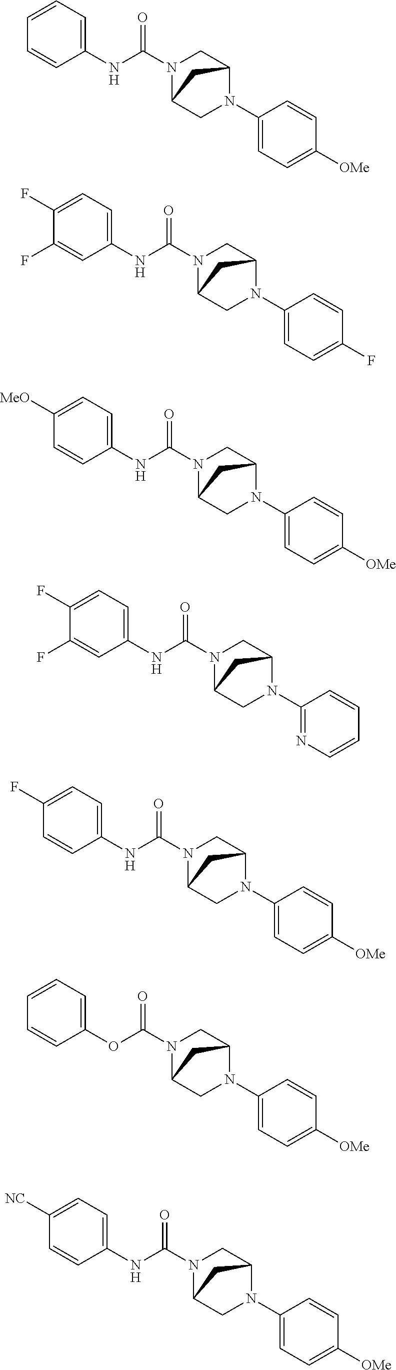

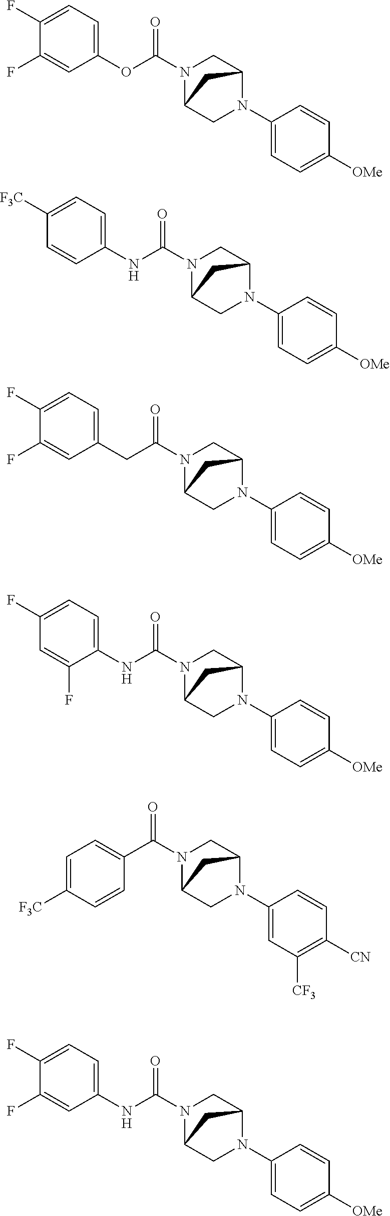 [SMILES: COc1ccc(N2C[C@H]3C[C@@H]2CN3C(=O)Oc2ccc(F)c(F)c2)cc1 ; COc1ccc(N2C[C@H]3C[C@@H]2CN3C(=O)Nc2ccc(C(F)(F)F)cc2)cc1 ; COc1ccc(N2C[C@H]3C[C@@H]2CN3C(=O)Cc2ccc(F)c(F)c2)cc1 ; COc1ccc(N2C[C@H]3C[C@@H]2CN3C(=O)Nc2ccc(F)cc2F)cc1 ; N#Cc1ccc(N2C[C@H]3C[C@@H]2CN3C(=O)c2ccc(C(F)(F)F)cc2)cc1C(F)(F)F ; COc1ccc(N2C[C@H]3C[C@@H]2CN3C(=O)Nc2ccc(F)c(F)c2)cc1]

or a pharmaceutically acceptable salt thereof.

In some embodiments, the glioblastoma is a DNA alkylating agent-resistant glioblastoma.

In some embodiments, the presently disclosed subject matter provides a method of inhibiting Brahma-related gene 1 (BRG1) bromodomain, the method comprising contacting a sample comprising BRG1 with a compound of Formula (I) and (II), subject to the proviso.

Accordingly, it is an object of the presently disclosed subject matter to provide compounds of Formula (I) and (II), or a pharmaceutical acceptable salt, solvate, amino acid conjugate, deuterium-labeled analogs, PROTAC-conjugate, related pharmaceutical formulations and methods of treating glioblastoma and inhibiting BRG1 bromodomain.

An object of the presently disclosed subject matter having been stated hereinabove, and which is achieved in whole or in part by the presently disclosed subject matter, other objects will become evident as the description proceeds hereinbelow.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. 1 shows the ability of bromodomain inhibitors to sensitize GBM cells to TMZ-induced cell death. MT330 GBM cells were treated with PFI-3 or the indicated bromodomain inhibitors alone (10 μM), and with TMZ (500 μM). Control cells were treated with vehicle (DMSO). Cell death was determined at 72 hr after treatment by a cell death ELISA that quantifies histone-complexed DNA fragments.

FIG. 2 shows that bromodomain inhibitors sensitize GBM cells to the antiproliferative action of TMZ. T98G GBM cells were treated with TMZ (500 μM), the bromodomain inhibitors (20 μM), and cell proliferation was determined by Incucyte live cell assays using cellular confluence method. The data was normalized to the calculated cell confluence at day 0. Control cells were treated with vehicle (DMSO).

FIG. 3 shows binding assays for bromodomain inhibitors to BRG1 bromodomain. MT330 cells expressing the BRG1 epitope tagged bromodomain construct were treated with either PFI-3 (30 μM), bromodomain inhibitors (30 μM), or DMSO for 3 hr. After heating over a temperature range from 44.5° C. to 55.6° C. for 5 min, the cells were lysed, placed on ice at 4° C. and then immunoblotted for BRG1 or actin.

FIG. 4 shows IV-129 is more potent than PFI-3 as a chemosensitizer to TMZ. Highly TMZ-resistant GBM cells (U87 and T98G) were treated with TMZ (500 μM) and either PFI-3, IV-129 or IV-191 (10 μM). After 72 hr, cell death was determined by a commercial cell death ELISA.

FIGS. 5A-5C demonstrate that bromodomain inhibitors increase survival after intracranial injection of GBM cells. MT330 cells (10⁶) were injected into the brains of NSG female mice. After tumors were identified by BLI (˜10 days post-injection), mice were IP injected with TMZ (60 mg/Kg body weight) or IV-129 (10 mg/Kg) alone or in combination 3 times per week (5 mice per group) for 2 weeks. FIG. 5A shows bioluminescent imaging of mice after luciferin injection at 20 days after treatment. FIG. 5B shows weight loss of mice at 20 days post-treatment as compared to vehicle treated mice that were not injected intracranially with tumor cells. FIG. 5C Kaplan-Meier analysis of the survival data (5 mice/group) of the brain tumor-bearing mice was performed.

FIGS. 6A-6C. show BRI IV-255 binds selectively to the BRG1 BRD and is a potent sensitizer to TMZ-induced cell death. FIG. 6A shows MT330 cells treated with TMZ (500 μM) and bromodomain inhibitors (10 μM). After 24 hr, cell death was determined by cell death ELISA, as shown in FIGS. 6B and 6C. For the CETSA, MT330 cells expressing the BRM (FIG. 6B), or BRG1 (FIG. 6C) the BRD construct were treated with bromodomain inhibitors (20 mM) and immunoblotted as indicated.

FIG. 7 shows that BRI YH-IV-255 sensitized BRM knockout GBM cells, but not BRG1 knockout cells to TMZ-induced cell death. BRG1 and BRM knockout MT330 cells were treated with TMZ (500 μM) and bromodomain inhibitors (10 μM). After 72 hr, cell death was determined by a cell death ELISA.

FIG. 8 shows PFI-3 sensitized GBM cells to DNA damage induced by Bleomycin. MT330 cells were treated with bleomycin alone (0.5 and 2 μM) or pretreated for 1 hr with PFI-3 (5 μM) followed by bleomycin treatment. After 1 hr of bleomycin treatment cell death, cells were fixed and immunostained for γH2Ax. Analysis of immunostaining was performed on at least 1,000 cells on the Lionheart FX imaging system.

FIG. 9 shows bromodomain inhibitors (YH-IV-129, YH-IV-255 and YH-IV-275) sensitized GBM cells to bleomycin-induced cell death. Parental and BRG1 knockout MT330 cells were treated with bleomycin (0.01 μM) and bromodomain inhibitors (10 μM). After 72 hr, cell death was determined by cell death ELISA.

FIG. 10 shows bromodomain inhibitors reduce tumor formation by intracranially injected GBM cells. MT330 cells (106) were injected into the brains of NSG female mice. After tumors were identified by BLI (˜10 days post-injection), mice were IP injected with TMZ (10 mg/Kg body weight), IV-255 or IV-275 (20 mg/Kg) alone or in combination 3 times per week (5 mice/group).

FIG. 11 shows bromodomain inhibitors enhance DNA damage in GBM cells induced by TMZ. LN229 cells were treated with TMZ (200 mM) in the presence or absence or the bromodomain inhibitors (20 mM of PFI-3, IV129, IV-255 or IV-275). After treatment for 24 hrs, cells were fixed and immunostained for gH2Ax. Analysis of immunostaining was performed on a laser confocal microscope.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, the presently disclosed subject matter provides a compound having the structure of Formula (I):

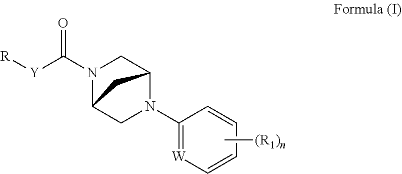

Wherein:
R represents alkyl, cycloalkyl, alkenyl, cycloalkyl, alkynyl, phenyl, aryl, heteroaryl or arylalkyl moiety having at most 12 carbon atoms, these moieties being optionally substituted by one or more substituents chosen from halogen atoms and the following functional groups: optionally esterified. etherified or protected hydroxy, alkoxy, hydroxyalkyl, alkenyloxy, alkynyloxy, trifluoromethyl, mercapto, halogens, cyano, nitro, acyl, acyloxy, aryl, esterified, amidified or salified carboxy, amino, mono-dialkylamino or hydrogen.
R₁=alkyl, alkoxy, hydroxy, esterified, etherified or protected hydroxy, amino, mono- or dialkylamino, halogens (F, Cl, Br, I), acyl, acyloxy, cyano, trifluoromethyl, nitro, esterified, amidified or salified carboxy, and hydrogen;
Y=absent, O, NH, CH₂;
W═CH, N;
n is an integer from 0 to 4;

In some embodiments, the compound of Formula (I) is selected from:

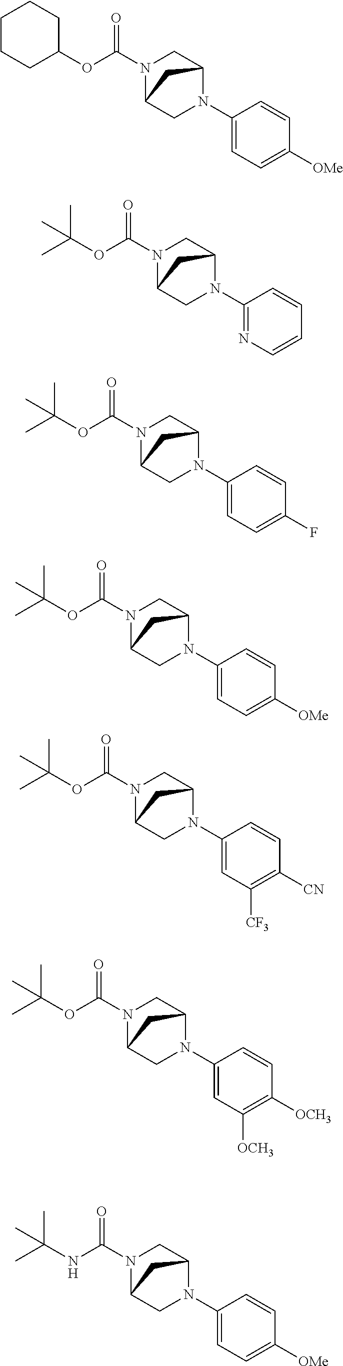

or a pharmaceutically acceptable salt thereof.

In some embodiments, the compound of Formula (I) has a structure of Formula (II):

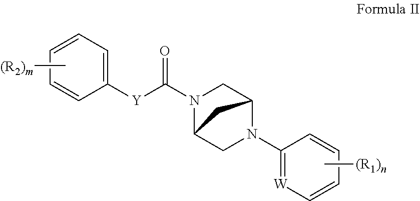

Wherein:
R₁=alkyl, alkoxy, hydroxy, esterified, etherified or protected hydroxy, amino, mono- or dialkylamino, halogens (F, Cl, Br, I), acyl, acyloxy, cyano, trifluoromethyl, nitro, esterified, amidified or salified carboxy, and hydrogen;
R²=alkyl, alkoxy, hydroxy, esterified, etherified or protected hydroxy, amino, mono- or dialkylamino, halogens (F, Cl, Br, I), acyl, acyloxy, cyano, trifluoromethyl, nitro, esterified, amidified or salified carboxy, and hydrogen;
Y=absent, O, NH, CH₂;
W═CH, N;
m or n is an integer from 0 to 4;

In some embodiments, the compound of Formula (II) is selected from:

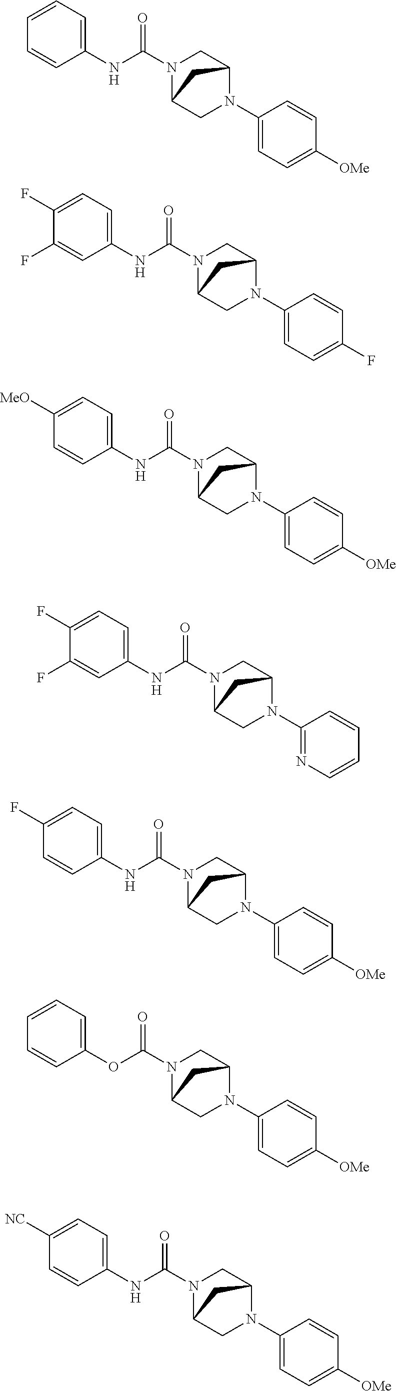

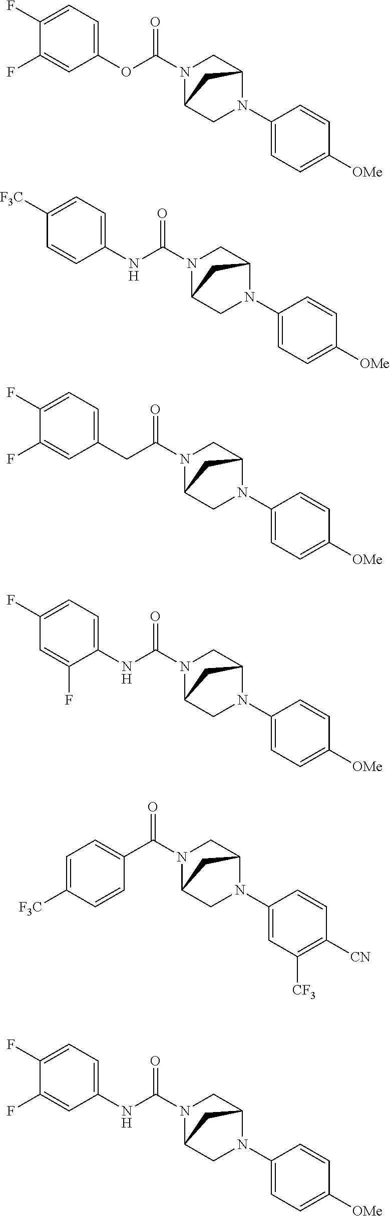 [SMILES: COc1ccc(N2C[C@H]3C[C@@H]2CN3C(=O)Oc2ccc(F)c(F)c2)cc1 ; COc1ccc(N2C[C@H]3C[C@@H]2CN3C(=O)Nc2ccc(C(F)(F)F)cc2)cc1 ; COc1ccc(N2C[C@H]3C[C@@H]2CN3C(=O)Cc2ccc(F)c(F)c2)cc1 ; COc1ccc(N2C[C@H]3C[C@@H]2CN3C(=O)Nc2ccc(F)cc2F)cc1 ; N#Cc1ccc(N2C[C@H]3C[C@@H]2CN3C(=O)c2ccc(C(F)(F)F)cc2)cc1C(F)(F)F ; COc1ccc(N2C[C@H]3C[C@@H]2CN3C(=O)Nc2ccc(F)c(F)c2)cc1]

or a pharmaceutically acceptable salt thereof.

In some embodiments, the glioblastoma is a DNA alkylating agent-resistant glioblastoma.

In some embodiments, the presently disclosed subject matter provides a method of inhibiting Brahma-related gene 1 (BRG1) bromodomain, the method comprising contacting a sample comprising BRG1 with a compound of Formula (I) and (II), subject to the proviso.

Accordingly, it is an object of the presently disclosed subject matter to provide cancer therapeutic compounds of Formula (I) and (II), which may include a pharmaceutical acceptable salt, solvate, amino acid conjugate, deuterium-labeled analogs, PROTAC-conjugate, related pharmaceutical formulations and methods of treating glioblastoma and inhibiting BRG1 bromodomain.

The cancer therapeutic of the present invention may comprise a pharmaceutically acceptable carrier and a therapeutically effective amount of a compound of Formula (I) or Formula (II).

The cancer therapeutic may be administered via various routes, including but not limited to oral, intravenous, intramuscular, subcutaneous, intraperitoneal, transdermal, and inhalation.

The choice of route of administration may depend on various factors, including the type of cancer, the stage of cancer, the patient's age and health condition, and the desired therapeutic effect.

In one embodiment of the invention, the cancer therapeutic is administered orally. The oral formulation may be in the form of tablets, capsules, granules, solutions, suspensions, or emulsions. The oral dosage range may be from about 10 mg to about 1000 mg per day, depending on the type and stage of cancer, the patient's age and health condition, and the desired therapeutic effect.

In another embodiment of the invention, the cancer therapeutic is administered intravenously. The intravenous formulation may be in the form of a solution or a suspension. The intravenous dosage range may be from about 1 mg to about 100 mg per day, depending on the type and stage of cancer, the patient's age and health condition, and the desired therapeutic effect. In yet another embodiment of the invention, the cancer therapeutic is administered subcutaneously. The subcutaneous formulation may be in the form of a solution or a suspension. The subcutaneous dosage range may be from about 10 mg to about 500 mg per day, depending on the type and stage of cancer, the patient's age and health condition, and the desired therapeutic effect.

In yet another embodiment of the invention, the cancer therapeutic is administered intraperitoneally. The intraperitoneal formulation may be in the form of a solution or a suspension. The intraperitoneal dosage range may be from about 10 mg to about 1000 mg per day, depending on the type and stage of cancer, the patient's age and health condition, and the desired therapeutic effect.

In still another embodiment of the invention, the cancer therapeutic is administered transdermally. The transdermal formulation may be in the form of a patch, ointment, lotion, serum, cream, or a gel. The transdermal dosage range may be from about 10 mg to about 500 mg per day, depending on the type and stage of cancer, the patient's age and health condition, and the desired therapeutic effect.

In yet another embodiment of the invention, the cancer therapeutic is administered via inhalation. The inhalation formulation may be in the form of an aerosol or a powder. The inhalation dosage range may be from about 1 mg to about 100 mg per day, depending on the type and stage of cancer, the patient's age and health condition, and the desired therapeutic effect.

The invention further relates to the use of the said combination for the treatment of various types of cancer. In another embodiment of the invention, the novel cancer therapeutic combines a compound of Formula (I) with at least one other active ingredient that exhibits potent anti-cancer properties. The active ingredient may be any compound that exhibits potent anti-cancer properties, including but not limited to small molecules, peptides, proteins, antibodies, nucleic acids, and their derivatives.

In a preferred embodiment, a compound of Formula (I) is combined with temozolomide. Temozolomide is an FDA-approved drug that is commonly used for the treatment of glioblastoma multiforme and other types of brain tumors. The drug works by causing DNA damage in cancer cells, leading to their death. However, temozolomide has limitations in terms of its efficacy and side effects, which limit its use as a monotherapy. Said combination product of a compound of Formula (I) and temozolomide may exhibit synergistic effects, leading to increased efficacy and decreased side effects compared to temozolomide alone.

“Pharmaceutical composition” refers to a formulation that comprises one or more active ingredients and one or more pharmaceutically acceptable carriers. The composition may be in any suitable form, including but not limited to tablets, capsules, powders, solutions, suspensions, emulsions, gels, creams, ointments, serums, lotions, patches, and injectable formulations. The choice of a pharmaceutical composition may depend on various factors, including the type and route of administration, the stability of the active ingredient, and the desired therapeutic effect. The composition may be prepared by any suitable method, including but not limited to blending, mixing, granulating, compressing, or lyophilizing.

“Pharmaceutically acceptable salt” refers to a salt form of a compound that is acceptable for use in pharmaceutical formulations. The salt form is typically prepared by reacting the compound with an appropriate acid or base to form a salt. The choice of the acid or base may depend on various factors, including the solubility, stability, and bioavailability of the salt.

Examples of pharmaceutically acceptable salts include, but are not limited to, hydrochloride, sulfate, citrate, tartrate, maleate, fumarate, mesylate, besylate, lactate, acetate, benzoate, succinate, and phosphate salts.

“Pharmaceutically acceptable carrier” refers to a substance or a combination of substances that are generally non-toxic and compatible with the active ingredient(s) of the cancer therapeutic. The carrier may be a solid, liquid, plasma, or a gas and may include, but is not limited to, excipients, diluents, binders, lubricants, disintegrants, fillers, and solvents. The choice of a pharmaceutically acceptable carrier may depend on various factors, including the type and route of administration, the stability of the active ingredient, and the desired therapeutic effect.

“Prodrug” refers to a pharmacologically inactive compound that is designed to be converted into an active drug in the body. Prodrugs may be advantageous for various reasons, including improving the pharmacokinetic properties of a drug, enhancing its bioavailability, and reducing its toxicity. Prodrugs may be converted into the active drug through various mechanisms, including hydrolysis, oxidation, reduction, or enzymatic cleavage.

“Therapeutically effective amount” refers to a quantity of the active ingredient(s) of the cancer therapeutic that exhibits the desired therapeutic effect in a patient. The amount may vary depending on various factors, including the type and stage of cancer, the patient's age and health condition, the route of administration, and the desired therapeutic effect. A therapeutically effective amount may be determined by one skilled in the art using routine experimentation, and may be expressed as a range or a specific value.

“Treating” or “treatment” refers to the administration of a therapeutic agent to a patient with the intention of alleviating, curing, or preventing a disease or medical condition. The term “treating” or “treatment” may also encompass prophylactic treatment, which is the administration of a therapeutic agent to prevent the onset or recurrence of a disease or medical condition.

### EXAMPLES

The following Examples are presented in order to more fully illustrate the preferred embodiments of the invention. They should in no way, however, be construed as limiting the broad scope of the invention. The following methods were used to conduct the experiments described in the Examples below:

### Example 1

### Synthesis of YH-IV-129 and YH-IV-181

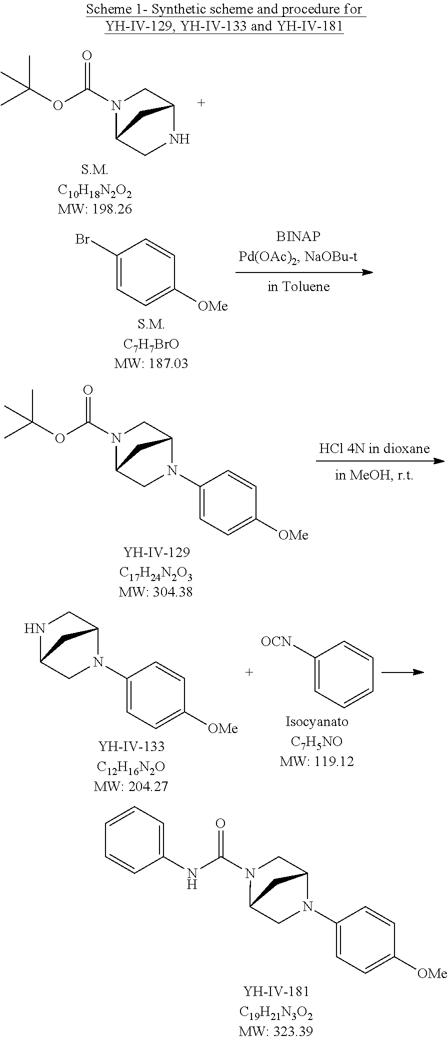

### YH-IV-129: (1R,4R)-tert-butyl 5-(4-methoxyphenyl)-2,5-diazabicyclo[2.2.1]heptane-2-carboxylate (C17H24N2O3)

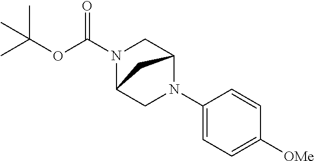 [SMILES: COc1ccc(N2C[C@H]3C[C@@H]2CN3C(=O)OC(C)(C)C)cc1]

To a mixture of (1R,4R)-tert-butyl 2,5-diazabicyclo[2.2.1]heptane-2-carboxylate (2.00 g, 0.010088 mol) in anhydrous toluene (50 mL) was added 1-bromo-4-methoxybenzene (3.77 g, 0.020176 mol), KOBu-t (1.70 g, 0.0151316 mol), Pd(OAc)₂ (0.113 g, 0.005044 mol), and (R)-(+)-2,2′-bis(diphenylphosphino)-1,1′-binaphthalene (BINAP, 0.314 g, 0.005044 mol) at room temperature under the argon atmosphere. The resulting reaction mixture was heated at reflux for 3 hours under argon. After the end of the reaction was established by TLC, the reaction was quenched by water, extracted with ethyl acetate. The organic layer was dried with MgSO₄, filtered, and concentrated under vacuum. The product was purified by a silica gel column using hexanes and ethyl acetate (2:1) as eluent to afford 1.76 g (57.3%) of the desired compound as pinkish solid.

¹HNMR (400 MHz, cdcl₃) δ 6.86-6.82 (m, 2H, ArH), 6.54-6.50 (m, 2H, ArH), 4.60-4.46 (br s, 1H, CH), 4.31 (br s, 1H, CH), 3.76 (s, 3H, CH₃O), 3.58-3.50 (m, 1H, CH), 3.43-3.32 (m, 2H, CH), 3.16-3.04 (m, 1H, CH), 1.98-1.86 (m, 2H, CH), 1.44 (s, 3H, CH₃), 1.39 (s, 6H, 2×CH₃).

Mass (ESI, Negative): [M−H]⁻; (ESI, Positive): [M+Na]⁺.

HRMS [C₁₇H₂₅N₂O₃+]: calculated 305.1865, found 305.1864 [M+H]⁺. Purity: 100.0% (HPLC).

### YH-IV-133: (1R,4R)-2-(4-methoxyphenyl)-2,5-diazabicyclo[2.2.1]heptane (C12H16N2O)

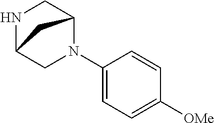 [SMILES: COc1ccc(N2C[C@H]3C[C@@H]2CN3)cc1]

To a solution of (1R,4R)-tert-butyl 5-(4-methoxyphenyl)-2,5-diazabicyclo[2.2.1]heptane-2-carboxylate (0.50 g, 0.0016427 mol) in methanol (10 mL), which was cooled in an ice water bath under an argon atmosphere, was added 4N HCl in dioxane (3.70 mL, 0.014777 mol). The resulting reaction mixture was allowed to stir 2-3 hours at room temperature under argon. After the end of the reaction was established by TLC, the reaction was concentrated under vacuum to afford 040 g as yellowish solid. The product was used for the next step without further purification.

¹HNMR (400 MHz, dmso-d₆) δ 6.88-6.80 (m, 2H, ArH), 6.62-6.58 (m, 2H, ArH), 4.70-4.66 (m, 1H, CH), 4.53-4.50 (s, 1H, CH), 4.31 (br s, 1H, CH), 3.66 (s, 3H, CH₃O), 3.64-3.55 (m, 1H, CH), 3.52-3.30 (m, 2H, CH and NH), 3.07-3.00 (m, 1H, CH), 2.10-2.06 (m, 1H, CH), 2.02-1.99 (m, 2H, CH).

HRMS [C₁₂H₁₇N₂O⁺]: calculated 205.1341, found 205.1346. Purity: 98.52% (HPLC).

### YH-IV-181: (1R,4R)-5-(4-methoxyphenyl)-N-phenyl-2,5-diazabicyclo[2.2.1]heptane-2-carboxamide (C19H21N3O2)

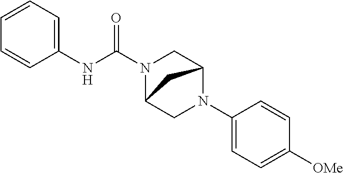 [SMILES: COc1ccc(N2C[C@H]3C[C@@H]2CN3C(=O)Nc2ccccc2)cc1]

To a solution of (1R,4R)-2-(4-methoxyphenyl)-2,5-diazabicyclo[2.2.1]heptane (0.117 g, 0.000575 mol) in 10 mL of anhydrous Dichloromethane (DCM) was added Et3N (0.29 g, 0.002875 mol) and isocyanatobenzene (0.137 g, 0.00115 mol), which was cooled in an ice bath under the argon atmosphere. The resulting reaction mixture was stirred and was homogenous at room temperature under argon for 3-4 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (9:1 to 4:1) as eluent to afford 0.53 g (82.3%) of the desired compound as white solid.

¹HNMR (400 MHz, DMSO₆) δ 8.21 (m, 1H, NH), 7.45 (d, J=8.0 Hz, 2H, ArH), 7.18 (t, J=8.2 Hz, 2H, ArH), 6.88 (t, J=8.0 Hz, 1H, ArH), 6.81-6.77 (m, 2H, ArH), 6.58-6.66 (m, 2H, ArH), 4.68 (s, 1H, CH), 4.48 (s, 1H, CH), 3.64 (s, 3H, OCH₃), 3.40 (s, 2H, CH), 3.43-3.38 (m, 1H, CH), 2.97-2.94 (m, 1H, CH), 1.98-1.97 (m, 1H, CH), 1.95-1.89 (m, 1H, CH).

Mass (ESI, Negative): [M−H]⁻; (ESI, Positive): [M+H]⁺.

HRMS [C₁₉H₂₂N₃O₂⁺]: calculated 324.1712, found 324.1704. Purity: 95.44% (HPLC).

### Example 2

### Synthesis of YH-IV-185

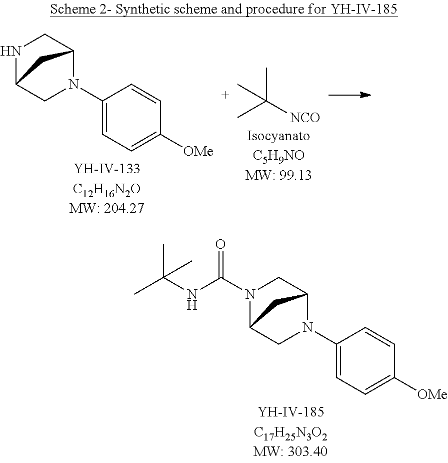

### YH-IV-185: (1R,4R)-N-(tert-butyl)-5-(4-methoxyphenyl)-2,5-diazabicyclo[2.2.1]heptane-2-carboxamide (C17H25N3O2)

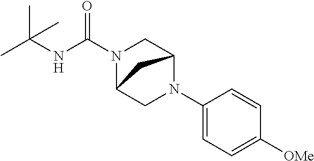 [SMILES: COc1ccc(N2C[C@H]3C[C@@H]2CN3C(=O)NC(C)(C)C)cc1]

To a solution of (1R,4R)-2-(4-methoxyphenyl)-2,5-diazabicyclo[2.2.1]heptane (0.117 g, 0.000575 mol) in anhydrous DCM (10 mL) was added Et3N (0.29 g, 0.002875 mol) and Tert-butyl isocyanate (0.114 g, 0.00115 mol), which was cooled in an ice bath under the argon atmosphere. The resulting reaction mixture was stirred and was homogenous at room temperature under argon for 3-4 hours. After the end of the reaction was established by TLC, the reaction was quenched by water, extracted with ethyl acetate. The organic layer was dried with MgSO₄, filtered, and concentrated under vacuum. The product was purified by a silica gel column using hexanes and ethyl acetate (1:1) as eluent to afford 0.158 g (85.0%) of the desired compound as white solid.

¹HNMR (400 MHz, DMSO₆) δ 6.80-6.76 (m, 2H, ArH), 6.53-6.50 (m, 2H, ArH), 5.40 (s, 1H, NH), 4.51 (s, 1H, CH), 4.38 (s, 1H, CH), 3.64 (s, 3H, OCH₃), 3.47-3.44 (m, 1H, CH), 3.21 (s, 2H, CH), 2.84-2.81 (m, 1H, CH), 1.87-1.85 (m, 1H, CH), 1.79-1.76 (m, 1H, CH), 1.19 (s, 9H, 3×CH₃).

Mass (ESI, Negative): [M−H]⁻; (ESI, Positive): [M+H]⁺.

HRMS [C₁₇H₂₆N₃O₂⁺]: calculated 304.2025, found 303.2017. Purity: % (HPLC).

### Example 3

### Synthesis of YH-IV-189

### YH-IV-189: tert-butyl 4-(4-methoxyphenyl)piperazine-1-carboxylate (C16H24N2O3)

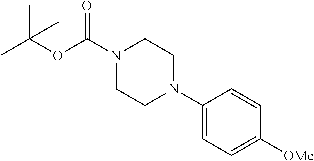 [SMILES: COc1ccc(N2CCN(C(=O)OC(C)(C)C)CC2)cc1]

¹HNMR (400 MHz, DMSO₆) δ 6.97-6.95 (m, 2H, ArH), 6.88-6.85 (m, 2H, ArH), 3.78 (s, 3H, OCH₃), 3.62 (br s, 4H, 2×CH₃), 3.05 (br s, 4H, 2×CH₃), 1.49 (s, 9H, 3×CH₃).

Mass (ESI, Negative): [M−H]⁻; (ESI, Positive): [M+H]⁺.

HRMS [C₁₆H₂₅N₂O₃⁺]: calculated 293.1865, found 293.1857. Purity: 74.85% (HPLC).

### Example 4

### Synthesis of YH-IV-211

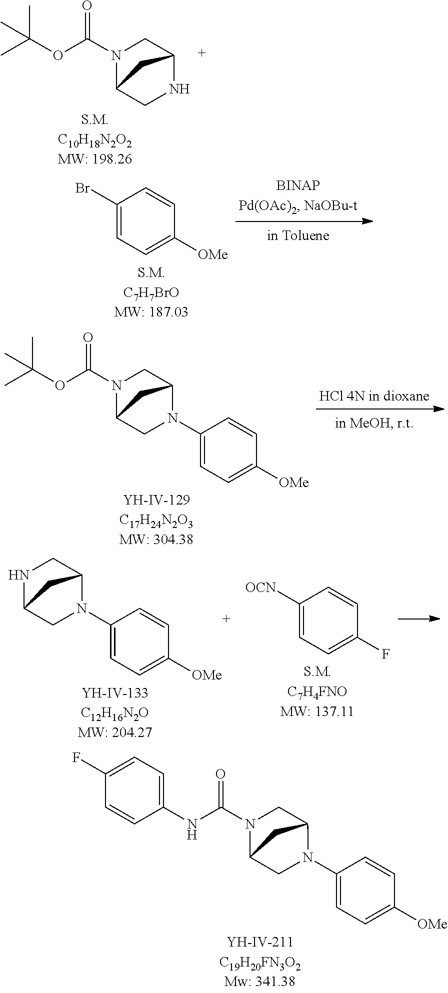

### YH-IV-211: (1R,4R)-N-(4-fluorophenyl)-5-(4-methoxyphenyl)-2,5-diazabicyclo[2.2.1]heptane-2-carboxamide (C19H20FN3O2)

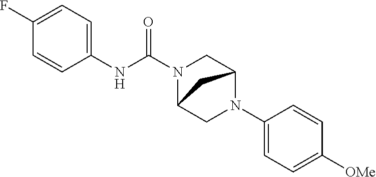 [SMILES: COc1ccc(N2C[C@H]3C[C@@H]2CN3C(=O)Nc2ccc(F)cc2)cc1]

To a solution of (1R,4R)-2-(4-methoxyphenyl)-2,5-diazabicyclo[2.2.1]heptane (0.2 g, 0.0009791 mol) in anhydrous DCM (10 mL) was added Et3N (0.495 g, 0.004895 mol) and 1-fluoro-4-isocyanatobenzene (0.268 g, 0.001958 mol), which was cooled in an ice bath under the argon atmosphere. The resulting reaction mixture was stirred and was homogenous at room temperature under argon for 3-4 hours. After the end of the reaction was established by TLC, the reaction was quenched by water, extracted with ethyl acetate. The organic layer was dried with MgSO₄, filtered, and concentrated under vacuum. The product was purified by a silica gel column using hexanes and ethyl acetate (1:1) as eluent to afford 0.167 g (50.0%) of the desired compound as off-white solid.

¹HNMR (400 MHz, DMSO₆) δ 8.26 (s, 1H, NH), 7.47-7.42 (m, 2H, ArH), 7.05-7.00 (m, 2H, ArH), 6.81-6.77 (m, 2H, ArH), 6.57-6.55 (m, 2H, ArH), 4.67 (s, 1H, CH), 4.48 (s, 1H, CH), 3.64 (s, 3H, OCH₃), 3.55-3.53 (m, 1H, CH), 3.41 (s, 2H, CH), 2.96-2.93 (m, 1H, CH), 1.98-1.94 (m, 1H, CH), 1.91-1.88 (m, 1H, CH).

Mass (ESI, Negative): [M−H]⁻; (ESI, Positive): [M+H]⁺.

HRMS [C₁₉H₂₁FN₃O₂⁺]: calculated 342.1618, found 342.1620. Purity: 97.70% (HPLC).

### Example 5

### Synthesis of YH-IV-215

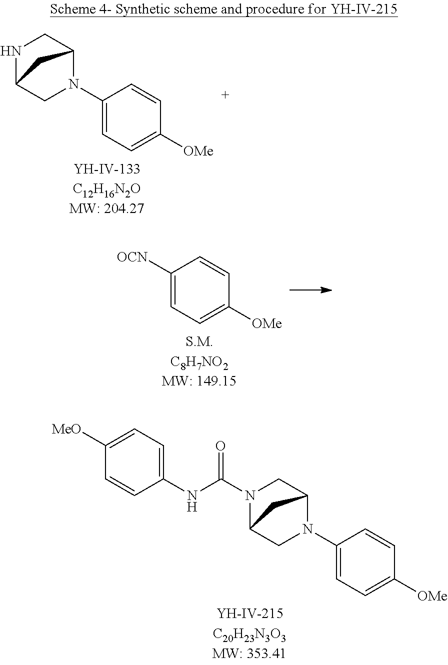

### YH-IV-215: (1R,4R)-N,5-bis(4-methoxyphenyl)-2,5-diazabicyclo[12.2.1]heptane-2-carboxamide (C20H23N3O3)

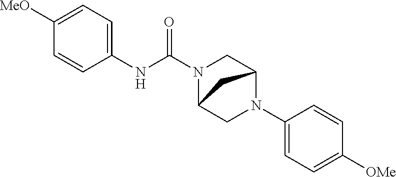 [SMILES: COc1ccc(NC(=O)N2C[C@H]3C[C@@H]2CN3c2ccc(OC)cc2)cc1]

To a solution of (1R,4R)-2-(4-methoxyphenyl)-2,5-diazabicyclo[12.2.1]heptane (0.2 g, 0.0009791 mol) in anhydrous DCM (10 mL) was added Et3N (0.495 g, 0.004895 mol) and 1-isocyanato-4-methoxybenzene (0.292 g, 0.001958 mol), which was cooled in an ice bath under the argon atmosphere. The resulting reaction mixture was stirred and was homogenous at room temperature under argon for 3-4 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and methanol (19:1) as eluent to afford 0.208 g (60.0%) of the desired compound as off-white solid.

¹ HNMR (400 MHz, DMSO₆) δ 8.06 (s, 1H, NH), 7.32-7.30 (m, 2H, ArH), 6.81-6.77 (m, 4H, ArH), 6.57-6.55 (m, 2H, ArH), 4.65 (s, 1H, CH), 4.47 (s, 1H, CH), 3.66 (s, 3H, OCH₃), 3.64 (s, 3H, OCH₃), 3.55-3.53 (m, 1H, CH), 3.37 (s, 2H, CH), 2.95-2.93 (m, 1H, CH), 1.96-1.94 (m, 1H, CH), 1.90-1.88 (m, 1H, CH).

Mass (ESI, Negative): [M−H]⁻; (ESI, Positive): [M+H]⁺.

HRMS [C₂₀H₂₄N₃O₃⁺]: calculated 354.1818, found 354.1806. Purity: 96.04% (HPLC).

### Example 6

### Synthesis of YH-IV-231

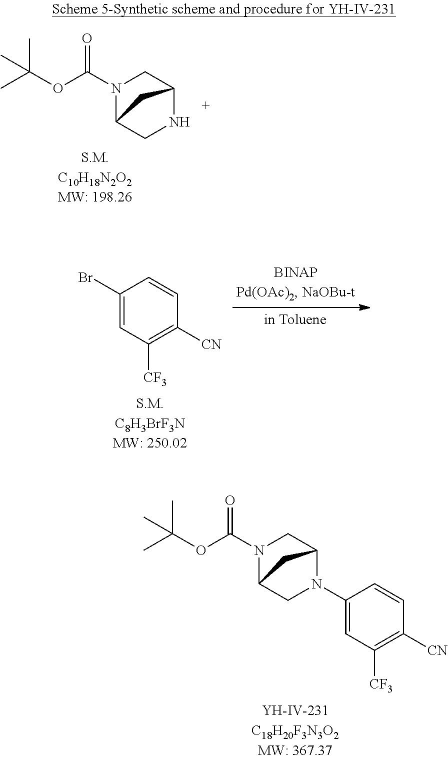 [SMILES: CC(C)(C)OC(=O)N1C[C@H]2C[C@@H]1CN2 ; N#Cc1ccc(Br)cc1C(F)(F)F ; CC(C)(C)OC(=O)N1C[C@H]2C[C@@H]1CN2c1ccc(C#N)c(C(F)(F)F)c1]

### YH-IV-231: (1R,4R)-tert-butyl 5-(4-cyano-3-(trifluoromethyl)phenyl)-2,5-diazabicyclo[2.2.1]heptane-2-carboxylate (C18H20F3N3O2)

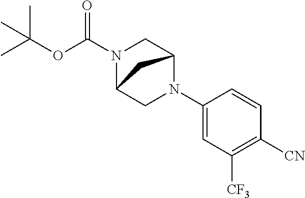 [SMILES: CC(C)(C)OC(=O)N1C[C@H]2C[C@@H]1CN2c1ccc(C#N)c(C(F)(F)F)c1]

To a mixture of (1R,4R)-tert-butyl 2,5-diazabicyclo[2.2.1]heptane-2-carboxylate (2.00 g, 0.010088 mol) in anhydrous toluene (50 mL) was added 4-bromo-2-(trifluoromethyl)benzonitrile (5.04 g, 0.020176 mol), KOBu-t (1.70 g, 0.0151316 mol), Pd(OAc)₂ (0.113 g, 0.005044 mol), and (R)-(+)-2,2′-bis(diphenylphosphino)-1,1′-binaphthalene (BINAP, 0.314 g, 0.005044 mol) at room temperature under the argon atmosphere. The resulting reaction mixture was heated at reflux for 3 hours under argon. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (19:1 to 9:1) as eluent to afford 1.08 g (29.1%) of the desired compound as light brown solid.

¹HNMR (400 MHz, cdcl₃) δ 6.86-6.82 (m, 2H, ArH), 6.54-6.50 (m, 2H, ArH), 4.60-4.46 (br s, 1H, CH), 4.31 (br s, 1H, CH), 3.76 (s, 3H, CH₃O), 3.58-3.50 (m, 1H, CH), 3.43-3.32 (m, 2H, CH), 3.16-3.04 (m, 1H, CH), 1.98-1.86 (m, 2H, CH), 1.44 (s, 3H, CH₃), 1.39 (s, 6H, 2×CH₃).

Mass (ESI, Negative): [M−H]⁻; (ESI, Positive): [M+Na]⁺.

HRMS [C₁₈H₂₁F₃N₃O₂⁺]: calculated 368.1586, found 368.1586 [M+H]⁺. Purity: 99.9% (HPLC).

### Example 7

### Synthesis of YH-IV-235

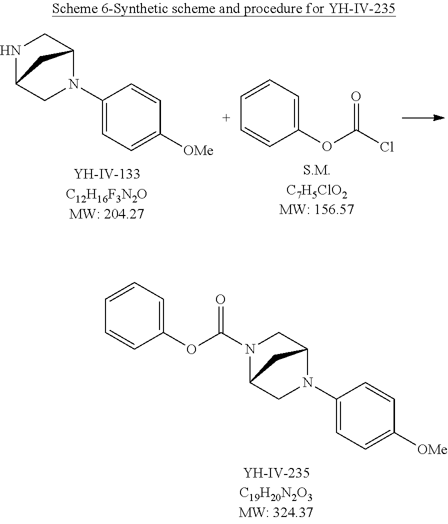 [SMILES: COc1ccc(N2C[C@H]3C[C@@H]2CN3)cc1 ; O=C(Cl)Oc1ccccc1 ; COc1ccc(N2C[C@H]3C[C@@H]2CN3C(=O)Oc2ccccc2)cc1]

### YH-IV-235:

### (1R,4R)-phenyl 5-(4-methoxyphenyl)-2,5-diazabicyclo[2.2.1]heptane-2-carboxylate (C19H20N2O3)

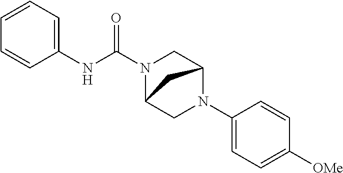 [SMILES: COc1ccc(N2C[C@H]3C[C@@H]2CN3C(=O)Nc2ccccc2)cc1]

To a solution of (1R,4R)-2-(4-methoxyphenyl)-2,5-diazabicyclo[2.2.1]heptane (0.20 g, 0.0009197 mol) in anhydrous DCM (10 mL) was added Et3N (0.495 g, 0.004895 mol) and phenyl carbonochloridate (0.307 g, 0.001958 mol), which was cooled in an ice bath under the argon atmosphere. The resulting reaction mixture was stirred and was homogenous at room temperature under argon for 5-6 hours. After the end of the reaction was established by TLC, the reaction was quenched by water, extracted with ethyl acetate. The organic layer was dried with MgSO₄, filtered, and concentrated under vacuum. The product was purified by a silica gel column using hexanes and ethyl acetate (1:1) as eluent to afford 0.28 g (88.0%) of the desired compound as pinkish solid.

¹HNMR (400 MHz, DMSO₆) δ 7.38-7.33 (m, 2H, ArH), 7.22-7.15 (m, 1H, ArH), 7.13-7.10 (m, 1H, ArH), 7.06-7.04 (m, 1H, ArH), 6.83-6.79 (m, 2H, ArH), 6.62-6.58 (m, 2H, ArH), 4.70 (s, 1H, CH), 4.53-4.48 (m, 1H, CH), 3.66 (s, 3H, OCH₃), 3.64-3.56 (m, 1H, CH), 3.48-3.39 (m, 1H, CH), 3.33-3.31 (m, 1H, CH), 3.13-3.01 (m, 1H, CH), 2.05-1.98 (m, 2H, CH).

Mass (ESI, Negative): [M−H]⁻; (ESI, Positive): [M+H]⁺.

HRMS [C₁₉H₂₁N₂O₃⁺]: calculated 325.1552, found 325.1552. Purity: 96.04% (HPLC).

HRMS [C₁₉H₁₉N₂O₃⁻]: calculated 323.1396, found 3223.1407. Purity: 96.04% (HPLC).

### Example 8

### Synthesis of YH-IV-239

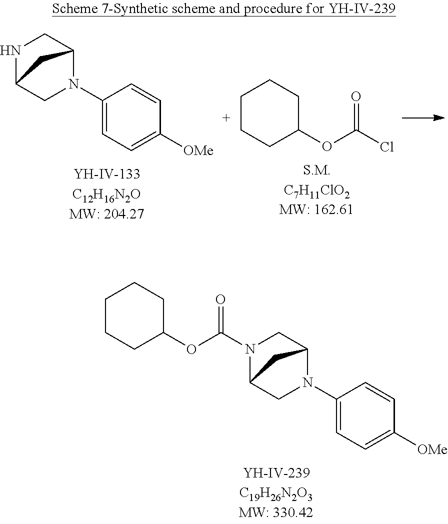 [SMILES: COc1ccc(N2C[C@H]3C[C@@H]2CN3)cc1 ; O=C(Cl)OC1CCCCC1 ; COc1ccc(N2C[C@H]3C[C@@H]2CN3C(=O)OC2CCCCC2)cc1]

### YH-IV-239:

### (1R,4R)-cyclohexyl 5-(4-methoxyphenyl)-2,5-diazabicyclo[2.2.1]heptane-2-carboxylate (C19H26N2O3)

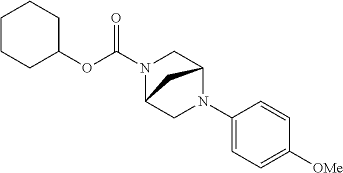 [SMILES: COc1ccc(N2C[C@H]3C[C@@H]2CN3C(=O)OC2CCCCC2)cc1]

To a solution of (1R,4R)-2-(4-methoxyphenyl)-2,5-diazabicyclo[2.2.1]heptane (0.20 g, 0.0009197 mol) in anhydrous DCM (10 mL) was added Et3N (0.495 g, 0.004895 mol) and cyclohexyl carbonochloridate (0.318 g, 0.001958 mol), which was cooled in an ice bath under the argon atmosphere. The resulting reaction mixture was stirred and was homogenous at room temperature under argon for 5-6 hours. After the end of the reaction was established by TLC, the reaction was quenched by water, extracted with ethyl acetate. The organic layer was dried with MgSO₄, filtered, and concentrated under vacuum. The product was purified by a silica gel column using hexanes and ethyl acetate (1:1) as eluent to afford 0.31 g (95.9%) of the desired compound as white solid.

¹HNMR (400 MHz, DMSO₆) δ 6.79-6.79 (m, 2H, ArH), 6.55-6.52 (m, 2H, ArH), 4.51-4.39 (m, 3H, CH), 3.64 (s, 3H, OCH₃), 3.55-3.49 (m, 1H, CH), 3.33-3.22 (m, 2H, CH), 2.90-2.84 (m, 1H, CH), 1.91-1.88 (m, 2H, CH), 1.71-1.63 (m, 4H, CH), 1.51-1.28 (m, 6H, CH).

Mass (ESI, Negative): [M−H]⁻; (ESI, Positive): [M+H]⁺.

HRMS [C₁₉H₂₇N₂O₃⁺]: calculated 331.2022, found 331.2022. Purity: 96.16% (HPLC).

### Example 9

### Synthesis of YH-IV-243

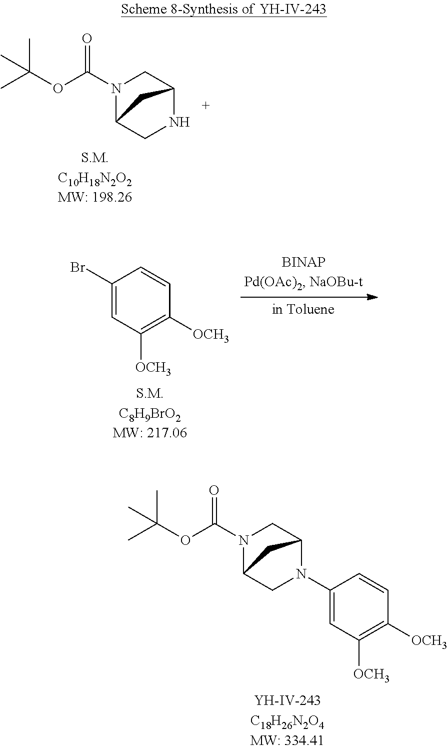 [SMILES: CC(C)(C)OC(=O)N1C[C@H]2C[C@@H]1CN2 ; COc1ccc(Br)cc1OC ; COc1ccc(N2C[C@H]3C[C@@H]2CN3C(=O)OC(C)(C)C)cc1OC]

### YH-IV-243: (1R,4R)-tert-butyl 5-(3,4-dimethoxyphenyl)-2,5-diazabicyclo[2.2.1]heptane-2-carboxylate (C18H26N2O4)

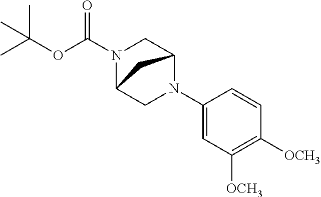 [SMILES: COc1ccc(N2C[C@H]3C[C@@H]2CN3C(=O)OC(C)(C)C)cc1OC]

To a mixture of (1R,4R)-tert-butyl 2,5-diazabicyclo[2.2.1]heptane-2-carboxylate (2.00 g, 0.010088 mol) in anhydrous toluene (50 mL) was added 4-bromo-1,2-dimethoxybenzene (4.38 g, 0.020176 mol), KOBu-t (1.70 g, 0.0151316 mol), Pd(OAc)₂ (0.113 g, 0.005044 mol), and (R)-(+)-2,2′-bis(diphenylphosphino)-1,1′-binaphthalene (BINAP, 0.314 g, 0.005044 mol) at room temperature under the argon atmosphere. The resulting reaction mixture was heated at reflux for 3 hours under argon. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (9:1) as eluent to afford 2.53 g (75.1%) of the desired compound as pinkish solid.

¹HNMR (400 MHz, cdcl₃) δ 6.78 (d, J=8.4 Hz, 1H, ArH), 6.25 (d, J=2.4 Hz, 1H, ArH), 6.06 (dd, J=8.4 Hz, J=2.4 Hz, 1H, ArH), 4.43-4.34 (m, 2H, CH), 3.72 (s, 3H, CH₃O), 3.63 (s, 3H, CH₃O), 3.53-3.48 (m, 1H, CH), 3.29-3.18 (m, 2H, CH), 2.93-2.22 (m, 1H, CH), 1.88-1.82 (m, 2H, CH), 1.37 (s, 3H, CH₃), 1.32 (s, 6H, 2×CH₃).

Mass (ESI, Negative): [M−H]⁻; (ESI, Positive): [M+Na]⁺.

HRMS [C₁₈H₂₇N₂₀₄⁺]: calculated 335.1971, found [M+H]⁺. Purity: 99.44% (HPLC).

### Example 10

### Synthesis of YH-IV-247

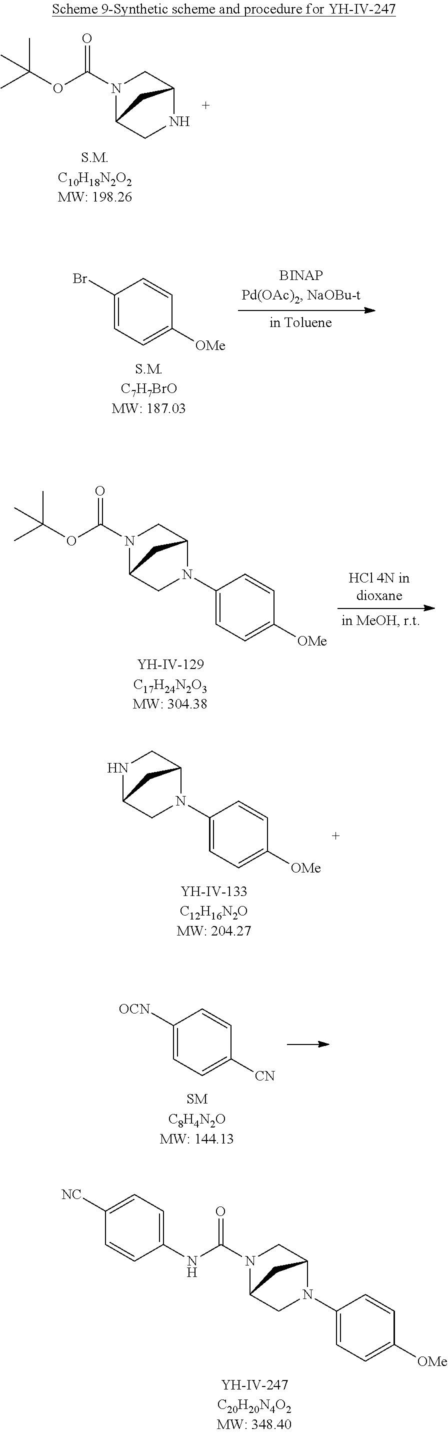

### YH-IV-247: (1R,4R)-N-(4-cyanophenyl)-5-(4-methoxyphenyl)-2,5-diazabicyclo[2.2.1]heptane-2-carboxamide (C20H20N4O2)

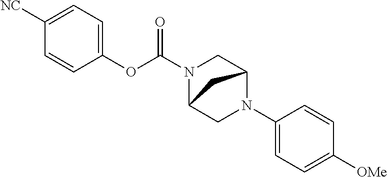 [SMILES: COc1ccc(N2C[C@H]3C[C@@H]2CN3C(=O)Oc2ccc(C#N)cc2)cc1]

To a solution of (1R,4R)-2-(4-methoxyphenyl)-2,5-diazabicyclo[2.2.1]heptane (0.2 g, 0.0009791 mol) in anhydrous DCM (10 mL) was added Et3N (0.495 g, 0.004895 mol) and 4-isocyanatobenzonitrile (0.28 g, 0.001958 mol), which was cooled in an ice bath under the argon atmosphere. The resulting reaction mixture was stirred and was homogenous at room temperature under argon for 6-8 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (9:1) as eluent to afford 0.306 g (90.0%) of the desired compound as off-white solid.

¹HNMR (400 MHz, DMSO₆) δ 8.70 (s, 1H, NH), 7.68-7.62 (m, 4H, ArH), 6.80-6.77 (m, 2H, ArH), 6.59-6.54 (m, 2H, ArH), 4.72 (s, 1H, CH), 4.50 (s, 1H, CH), 3.61 (s, 3H, OCH₃), 3.57-3.54 (m, 1H, CH), 3.46-3.43 (m, 2H, CH), 2.97-2.95 (m, 1H, CH), 1.98-1.96 (m, 1H, CH), 1.92-1.90 (m, 1H, CH).

Mass (ESI, Negative): [M−H]⁻; (ESI, Positive): [M+H]⁺.

HRMS [C₂₀H₂₁N₄O₂⁺]: calculated 349.1665, found 349.1665. Purity: 97.54% (HPLC).

HRMS [C₂₀H₁₉N₄O₂₁]: calculated 347.1508, found 347.1578. Purity: 97.54% (HPLC).

### Example 11

### Synthesis of YH-IV-251

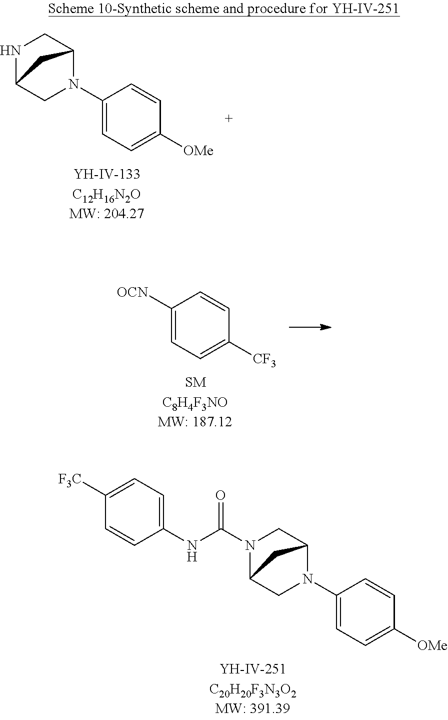

### YH-IV-251:

### (1R,4R)-5-(4-methoxyphenyl)-N-(4-(trifluoromethyl)phenyl)-2,5-diazabicyclo[2.2.1]heptane-2-carboxamide (C20H20F3N3O2)

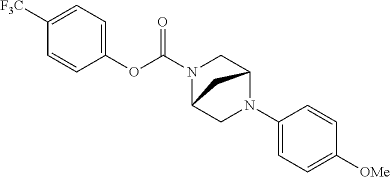 [SMILES: COc1ccc(N2C[C@H]3C[C@@H]2CN3C(=O)Oc2ccc(C(F)(F)F)cc2)cc1]

To a solution of (1R,4R)-2-(4-methoxyphenyl)-2,5-diazabicyclo[2.2.1]heptane (0.2 g, 0.0009791 mol) in anhydrous DCM (10 mL) was added Et3N (0.495 g, 0.004895 mol) and 1-isocyanato-4-(trifluoromethyl)benzene (0.28 g, 0.001958 mol), which was cooled in an ice bath under the argon atmosphere. The resulting reaction mixture was stirred and was homogenous at room temperature under argon for 6-8 hours. After the end of the reaction was established by TLC, the reaction was quenched by water, extracted with ethyl acetate. The organic layer was dried with MgSO₄, filtered, and concentrated under vacuum. The product was purified by a silica gel column using hexanes and ethyl acetate (1:1) as eluent to afford 0.34 g (88.8%) of the desired compound as off-white foam.

¹HNMR (400 MHz, DMSO₆) δ 8.61 (s, 1H, NH), 7.70 (d, J=8.8 Hz, 2H, ArH), 7.55 (d, J=8.8 Hz, 2H, ArH), 6.81-6.77 (m, 2H, ArH), 6.59-6.54 (m, 2H, ArH), 4.72 (s, 1H, CH), 4.50 (s, 1H, CH), 3.64 (s, 3H, OCH₃), 3.57-3.54 (m, 1H, CH), 3.46-3.40 (m, 2H, CH), 2.97-2.95 (m, 1H, CH), 1.98-1.96 (m, 1H, CH), 1.92-1.90 (m, 1H, CH).

Mass (ESI, Negative): [M−H]⁻; (ESI, Positive): [M+H]⁺.

HRMS [C₁₉H₂₁F₃N₃O₂⁺]: calculated 392.1586, found 392.1586. Purity: 99.66% (HPLC).

HRMS [C₁₉H₁₉F3N₃O₂⁺]: calculated 390.1429, found 390.1427. Purity: 99.66% (HPLC).

### Example 12

### Synthesis of YH-IV-255

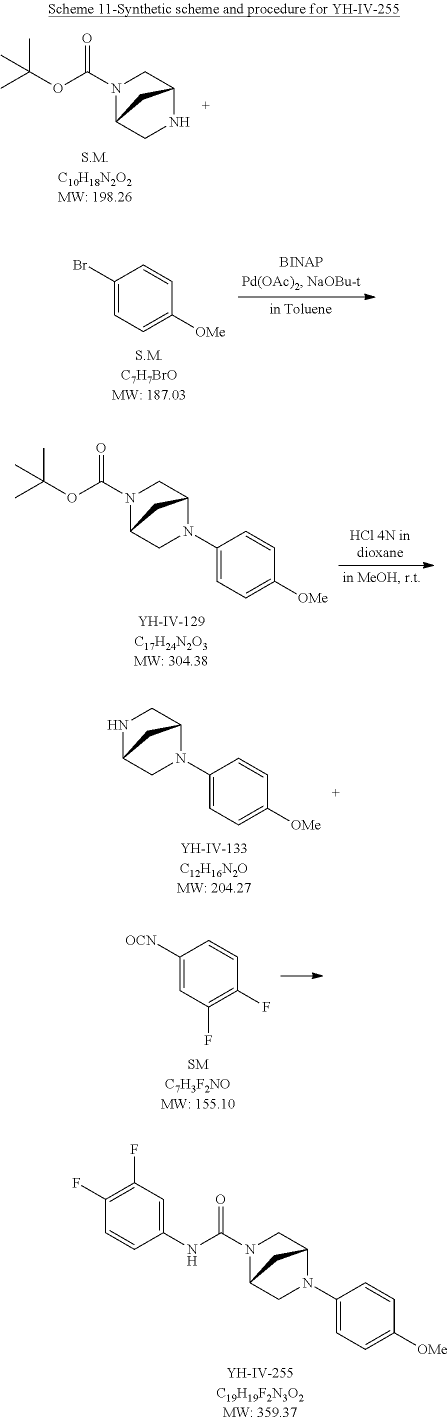

### YH-IV-255:

### (1R,4R)-N-(3,4-difluorophenyl)-5-(4-methoxyphenyl)-2,5-diazabicyclo[2.2.1]heptane-2-carboxamide (C19H19F2N3O2)

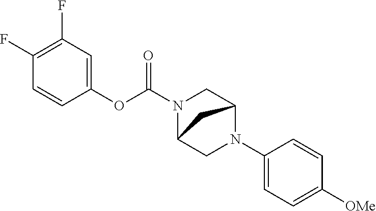 [SMILES: COc1ccc(N2C[C@H]3C[C@@H]2CN3C(=O)Oc2ccc(F)c(F)c2)cc1]

To a solution of (1R,4R)-2-(4-methoxyphenyl)-2,5-diazabicyclo[2.2.1]heptane (0.2 g, 0.0009791 mol) in anhydrous DCM (10 mL) was added Et3N (0.495 g, 0.004895 mol) and 1,2-difluoro-4-isocyanatobenzene (0.304 g, 0.001958 mol), which was cooled in an ice bath under the argon atmosphere. The resulting reaction mixture was stirred and was homogenous at room temperature under argon for 6-8 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (9:1 to 4:1) as eluent to afford 0.30 g (85%) of the desired compound as pinkish solid.

¹HNMR (400 MHz, DMSO₆) δ 8.43 (s, 1H, NH), 7.65-7.59 (m, 1H, ArH), 7.29-7.21 (m, 2H, ArH), 6.81-6.77 (m, 2H, ArH), 6.57-6.54 (m, 2H, ArH), 4.67 (s, 1H, CH), 4.49 (s, 1H, CH), 3.64 (s, 3H, OCH₃), 3.56-3.54 (m, 1H, CH), 3.42-3.37 (m, 2H, CH), 2.95-2.93 (m, 1H, CH), 1.98-1.96 (m, 1H, CH), 1.92-1.89 (m, 1H, CH).

Mass (ESI, Negative): [M−H]⁻; (ESI, Positive): [M+H]⁺.

HRMS [C₁₉H₂₀F₂N₃O₂⁺]: calculated 360.1525, found 360.1525. Purity: 97.44% (HPLC).

HRMS [C₁₉H₁₈F₂N₃O₂⁺]: calculated 358.1367, found 358.1369. Purity: 97.44% (HPLC).

### Example 13

### Synthesis of YH-IV-259

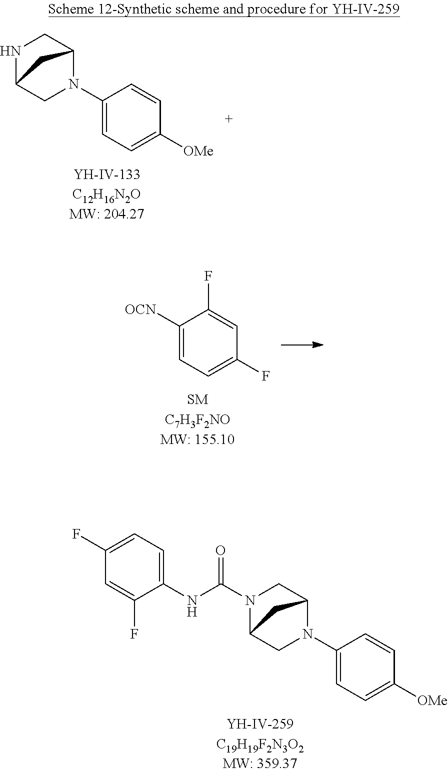

### YH-IV-259:

### (1R,4R)-N-(2,4-difluorophenyl)-5-(4-methoxyphenyl)-2,5-diazabicyclo[2.2.1]heptane-2-carboxamide (C19H19F2N3O2)

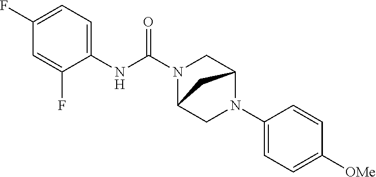 [SMILES: COc1ccc(N2C[C@H]3C[C@@H]2CN3C(=O)Nc2ccc(F)cc2F)cc1]

To a solution of (1R,4R)-2-(4-methoxyphenyl)-2,5-diazabicyclo[2.2.1]heptane (0.2 g, 0.0009791 mol) in anhydrous DCM (10 mL) was added Et3N (0.495 g, 0.004895 mol) and 2,4-difluoro-1-isocyanatobenzene (0.304 g, 0.001958 mol), which was cooled in an ice bath under the argon atmosphere. The resulting reaction mixture was stirred and was homogenous at room temperature under argon for 6-8 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (9:1 to 4:1) as eluent to afford 0.30 g (86.5%) of the desired compound as pinkish solid.

¹HNMR (400 MHz, DMSO₆) δ 8.04 (s, 1H, NH), 7.43-7.37 (m, 1H, ArH), 7.23-7.18 (m, 1H, ArH), 6.99-6.95 (m, 1H, ArH), 6.81-6.78 (m, 2H, ArH), 6.57-6.54 (m, 2H, ArH), 4.47 (s, 1H, CH), 4.47 (s, 1H, CH), 3.65 (s, 3H, OCH₃), 3.55-3.53 (m, 1H, CH), 3.39-3.34 (m, 2H, CH), 2.98-2.95 (m, 1H, CH), 1.98-1.96 (m, 1H, CH), 1.92-1.89 (m, 1H, CH).

Mass (ESI, Negative): [M−H]⁻; (ESI, Positive): [M+H]⁺.

HRMS [C₁₉H₂₀F₂N₃O₂⁺]: calculated 360.1525, found 360.1525. Purity: 97.44% (HPLC).

HRMS [C_{I9}H₁₈F₂N₃O₂⁺]: calculated 358.1367, found 358.1369. Purity: 97.44% (HPLC).

### Example 14

### Synthesis of YH-IV-267

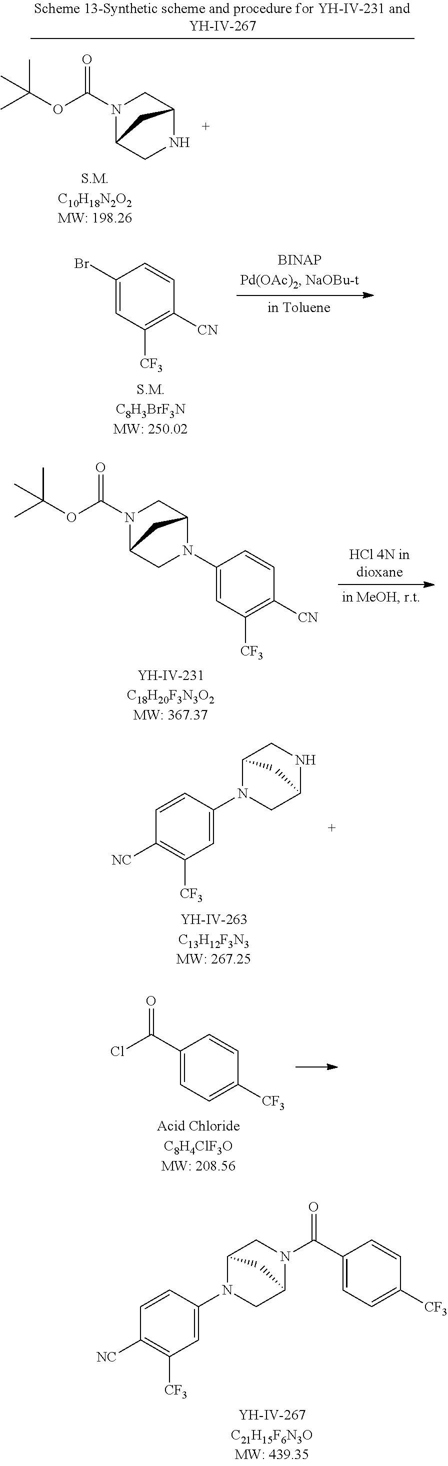 [SMILES: CC(C)(C)OC(=O)N1C[C@H]2C[C@@H]1CN2 ; N#Cc1ccc(Br)cc1C(F)(F)F ; CC(C)(C)OC(=O)N1C[C@H]2C[C@@H]1CN2c1ccc(C#N)c(C(F)(F)F)c1 ; N#Cc1ccc(N2C[C@H]3C[C@@H]2CN3)cc1C(F)(F)F ; O=C(Cl)c1ccc(C(F)(F)F)cc1 ; N#Cc1ccc(N2C[C@H]3C[C@@H]2CN3C(=O)c2ccc(C(F)(F)F)cc2)cc1C(F)(F)F]

### YH-IV-231: (1R,4R)-tert-butyl 5-(4-cyano-3-(trifluoromethyl)phenyl)-2,5-diazabicyclo[2.2.1]heptane-2-carboxylate (C18H20F3N3O2)

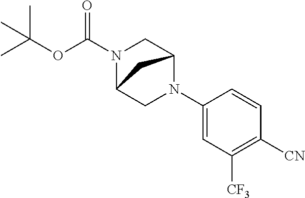 [SMILES: CC(C)(C)OC(=O)N1C[C@H]2C[C@@H]1CN2c1ccc(C#N)c(C(F)(F)F)c1]

To a mixture of (1R,4R)-tert-butyl 2,5-diazabicyclo[2.2.1]heptane-2-carboxylate (2.00 g, 0.010088 mol) in anhydrous toluene (50 mL) was added 4-bromo-2-(trifluoromethyl)benzonitrile (5.04 g, 0.020176 mol), KOBu-t (1.70 g, 0.0151316 mol), Pd(OAc)₂ (0.113 g, 0.005044 mol), and (R)-(+)-2,2′-bis(diphenylphosphino)-1,1′-binaphthalene (BINAP, 0.314 g, 0.005044 mol) at room temperature under the argon atmosphere. The resulting reaction mixture was heated at reflux for 3 hours under argon. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (19:1 to 9:1) as eluent to afford 1.08 g (29.1%) of the desired compound as light brown solid.

¹HNMR (400 MHz, cdcl₃) δ 6.86-6.82 (m, 2H, ArH), 6.54-6.50 (m, 2H, ArH), 4.60-4.46 (br s, 1H, CH), 4.31 (br s, 1H, CH), 3.76 (s, 3H, CH₃O), 3.58-3.50 (m, 1H, CH), 3.43-3.32 (m, 2H, CH), 3.16-3.04 (m, 1H, CH), 1.98-1.86 (m, 2H, CH), 1.44 (s, 3H, CH₃), 1.39 (s, 6H, 2×CH₃).

Mass (ESI, Negative): [M−H]⁻; (ESI, Positive): [M+Na]⁺.

HRMS [C₁₈H₂₁F₃N₃O₂⁺]: calculated 368.1586, found 368.1586 [M+H]⁺. Purity: 99.9% (HPLC).

### YH-IV-263: 4-((1R,4R)-2,5-diazabicyclo[2.2.1]heptan-2-yl)-2-(trifluoromethyl)benzonitrile (C13H12F3N3)

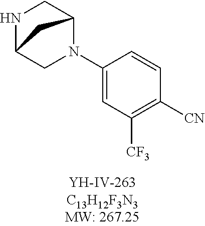 [SMILES: N#Cc1ccc(N2C[C@H]3C[C@@H]2CN3)cc1C(F)(F)F]

To a solution of (1R,4R)-tert-butyl 5-(4-cyano-3-(trifluoromethyl)phenyl)-2,5-diazabicyclo[2.2.1]heptane-2-carboxylate (0.62 g, 0.0022517 mol) in methanol (10 mL), which was cooled in an ice water bath under an argon atmosphere, was added 2N HCl in Et₂O (10 mL, 0.020265 mol). The resulting reaction mixture was allowed to stir 30 minutes at room temperature under argon. After the end of the reaction was established by TLC, the reaction was concentrated under vacuum to afford 0.60 g as yellowish solid. The product was used for the next step without further purification.

¹HNMR (400 MHz, DMSO₆) δ

Mass (ESI, Negative): [M−H]⁻; (ESI, Positive): [M+H]⁺.

HRMS [C₁₃H₁₃F₃N₃⁺]: calculated 268.1062, found 268.1062. Purity: % (HPLC).

HRMS [C₁₉H₁₁F₃N₃⁻]: calculated 266.0905, found 266.0905. Purity: % (HPLC).

### YH-IV-267: 2-(trifluoromethyl)-4-((1R,4R)-5-(4-(trifluoromethyl)benzoyl)-2,5-diazabicyclo[2.2.1]heptan-2-yl)benzonitrile (C21H15F6N3O)

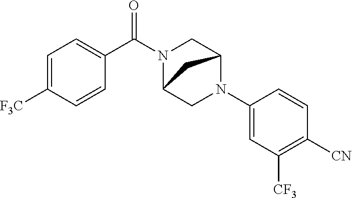 [SMILES: N#Cc1ccc(N2C[C@H]3C[C@@H]2CN3C(=O)c2ccc(C(F)(F)F)cc2)cc1C(F)(F)F]

To a solution of 4-((1S,4S)-2,5-diazabicyclo[2.2.1]heptan-2-yl)-2-(trifluoromethyl)benzonitrile (0.218 g, 0.0008166 mol) in anhydrous DCM (10 mL) was added Et3N (0.41 g, 0.004083 mol) and 4-(trifluoromethyl)benzoyl chloride (0.34 g, 0.0016332 mol), which was cooled in an ice bath under the argon atmosphere. The resulting reaction mixture was stirred and was homogenous at room temperature under argon for 5-6 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (9:1) as eluent to afford 0.304 g (80.0%) of the desired compound as yellowish foam.

¹HNMR (400 MHz, DMSO₆) δ 7.86-7.72 (m, 3H, ArH), 7.00-6.94 (m, 3H, ArH), 4.99-4.88 (m, 1H, CH), 4.42 (s, 1H, CH), 3.68-3.58 (m, 2H, CH), 3.43-3.23 (m, 2H, CH), 2.90-2.84 (m, 1H, CH), 2.13-1.93 (m, 2H, CH).

Mass (ESI, Negative): [M−H]⁻; (ESI, Positive): [M+H]⁺.

HRMS [C₂₁H₁₆F₆N₂O⁺]: calculated 440.1198, found 440.1198. Purity: % (HPLC).

### Example 15

### Synthesis of YH-IV-275

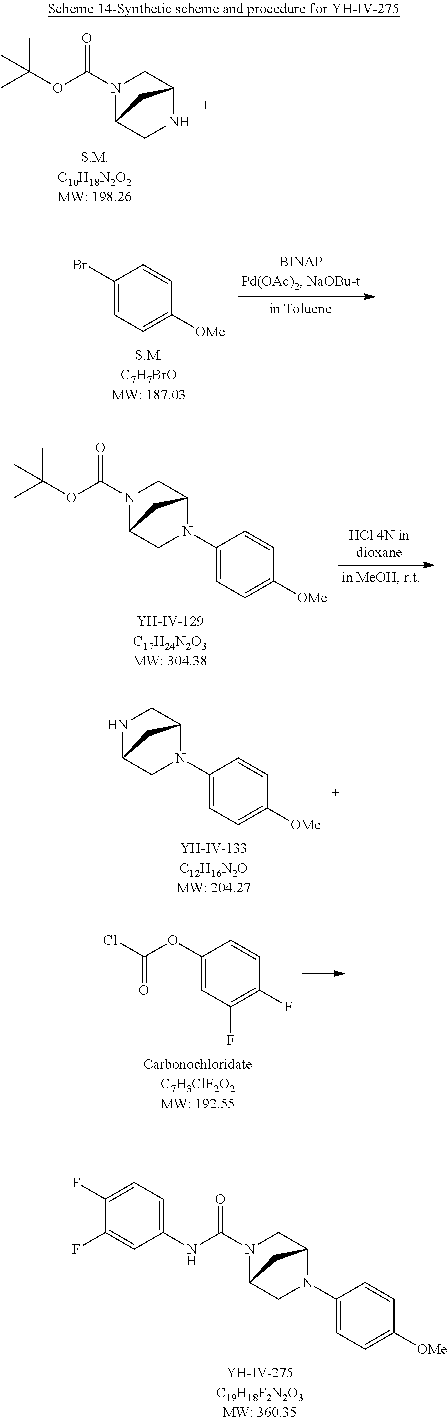 [SMILES: CC(C)(C)OC(=O)N1C[C@H]2C[C@@H]1CN2 ; COc1ccc(Br)cc1 ; COc1ccc(N2C[C@H]3C[C@@H]2CN3C(=O)OC(C)(C)C)cc1 ; COc1ccc(N2C[C@H]3C[C@@H]2CN3)cc1 ; O=C(Cl)Oc1ccc(F)c(F)c1 ; COc1ccc(N2C[C@H]3C[C@@H]2CN3C(=O)Nc2ccc(F)c(F)c2)cc1]

### YH-IV-271:

### 3,4-difluorophenyl carbonochloridate (C7H3ClF₂O₂)

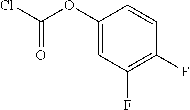 [SMILES: O=C(Cl)Oc1ccc(F)c(F)c1]

To a solution of 3,4-difluorophenol (0.39 g, 0.003 mmol), DIPEA (0.78 g, 0.006 mmol) in dry DCM was added dropwise a solution of triphosgene (0.89 g, 0.003 mmol) in dry DCM, which was cooled in an ice bath under an argon atmosphere. After addition, the resulting solution was stirred for 2-3 hours at room temperature. The above solution was cooled in an ice bath under argon, additional DIPAEA was added, and then went to the next step.

### YH-IV-275:

### (1R,4R)-3,4-difluorophenyl 5-(4-methoxyphenyl)-2,5-diazabicyclo [2.2.1]heptane-2-carboxylate (C19H18F2N2O3)

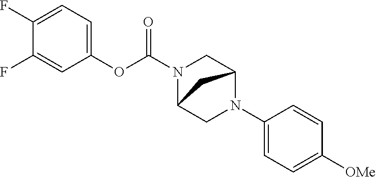 [SMILES: COc1ccc(N2C[C@H]3C[C@@H]2CN3C(=O)Oc2ccc(F)c(F)c2)cc1]

To a solution of (1R,4R)-2-(4-methoxyphenyl)-2,5-diazabicyclo[2.2.1]heptane (0.20 g, 0.0009197 mol) in anhydrous DCM (10 mL) was added Et3N (0.495 g, 0.004895 mol) and 3,4-difluorophenyl carbonochloridate (0.377 g, 0.001958 mol), which was cooled in an ice bath under the argon atmosphere. The resulting reaction mixture was stirred and was homogenous at room temperature under argon for 5-6 hours. After the end of the reaction was established by TLC, the reaction was quenched by water, extracted with ethyl acetate. The organic layer was dried with MgSO₄, filtered, and concentrated under vacuum. The product was purified by a silica gel column using hexanes and ethyl acetate (2:1) as eluent to afford 0.133 g (35.0%) of the desired compound as yellow solid.

¹HNMR (400 MHz, DMSO₆) δ 7.46-7.33 (m, 2H, ArH), 7.05-6.96 (m, 1H, ArH), 6.83-6.81 (m, 2H, ArH), 6.62-6.58 (m, 2H, ArH), 4.70 (s, 1H, CH), 4.55-4.49 (m, 1H, CH), 3.67 (s, 3H, OCH₃), 3.62-3.59 (m, 1H, CH), 3.48-3.39 (m, 1H, CH), 3.33-3.31 (m, 1H, CH), 3.18-3.02 (m, 1H, CH), 2.05-1.98 (m, 2H, CH).

Mass (ESI, Negative): [M−H]⁻; (ESI, Positive): [M+H]⁺.

HRMS [C₁₉H₂₁N₂O₃⁺]: calculated 325.1552, found 325.1552. Purity: 96.04% (HPLC).

HRMS [C₁₉H₁₉N₂O₃⁻]: calculated 323.1396, found 3223.1407. Purity: 96.04% (HPLC).

### Example 16

### Synthesis of YH-IV-291

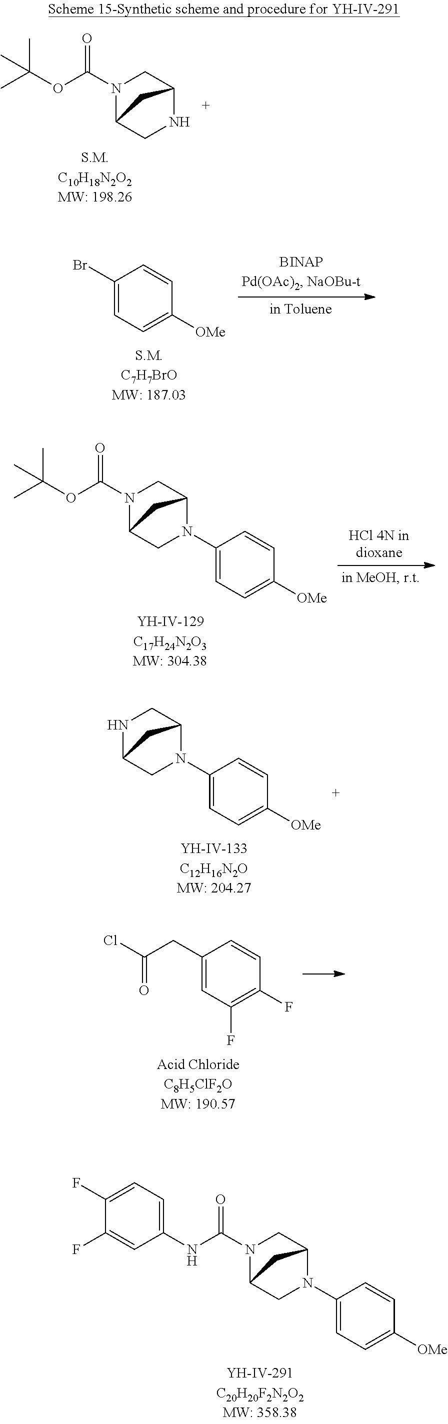 [SMILES: CC(C)(C)OC(=O)N1C[C@H]2C[C@@H]1CN2 ; COc1ccc(Br)cc1 ; COc1ccc(N2C[C@H]3C[C@@H]2CN3C(=O)OC(C)(C)C)cc1 ; COc1ccc(N2C[C@H]3C[C@@H]2CN3)cc1 ; O=C(Cl)Cc1ccc(F)c(F)c1 ; COc1ccc(N2C[C@H]3C[C@@H]2CN3C(=O)Nc2ccc(F)c(F)c2)cc1]

### YH-IV-291: 2-(3,4-difluorophenyl)-1-((1R,4R)-5-(4-methoxyphenyl)-2,5-diazabicyclo[2.2.1]heptan-2-yl)ethanone (C20H20F2N2O2)

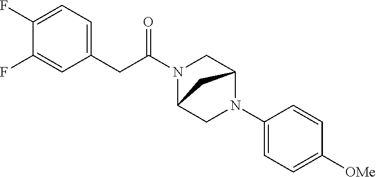 [SMILES: COc1ccc(N2C[C@H]3C[C@@H]2CN3C(=O)Cc2ccc(F)c(F)c2)cc1]

To a solution of (1R,4R)-2-(4-methoxyphenyl)-2,5-diazabicyclo[2.2.1]heptane (0.168 g, 0.00082135 mol) in anhydrous DCM (10 mL) was added Et3N (0.415 g, 0.0041067 mol) and 2-(3,4-difluorophenyl)acetyl chloride (0.157 g, 0.00082135 mol), which was cooled in an ice bath under the argon atmosphere. The resulting reaction mixture was stirred and was homogenous at room temperature under argon for 5-6 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (4:1) as eluent to afford 0.055 g (10.0%) of the desired compound as yellow solid.

¹HNMR (400 MHz, DMSO₆) δ 7.40-7.34 (m, 2H, ArH), 7.28-7.24 (m, 1H, ArH), 6.85-6.81 (m, 2H, ArH), 6.58-6.55 (m, 2H, ArH), 4.83 (s, 1H, CH), 4.56 (m, 1H, CH), 3.67 (s, 3H, OCH₃), 3.62-3.47 (m, 3H, CH), 3.40 (s, 2H, CH₂), 2.95-2.90 (m, 1H, CH), 2.05-2.03 (m, 1H, CH), 1.98-1.95 (m, 1H, CH).

Mass (ESI, Negative): [M−H]⁻; (ESI, Positive): [M+H]⁺.

HRMS [C₂₀H₂₁F₂N₂O₃⁺]: calculated 359.1571, found 359.1570. Purity: 95.83% (HPLC).

HRMS [C₂₀H₁₉F₂N₂O₃⁻]: calculated 357.1415, found. Purity: 95.83% (HPLC).

### Example 17

### Synthesis of YH-IV-295

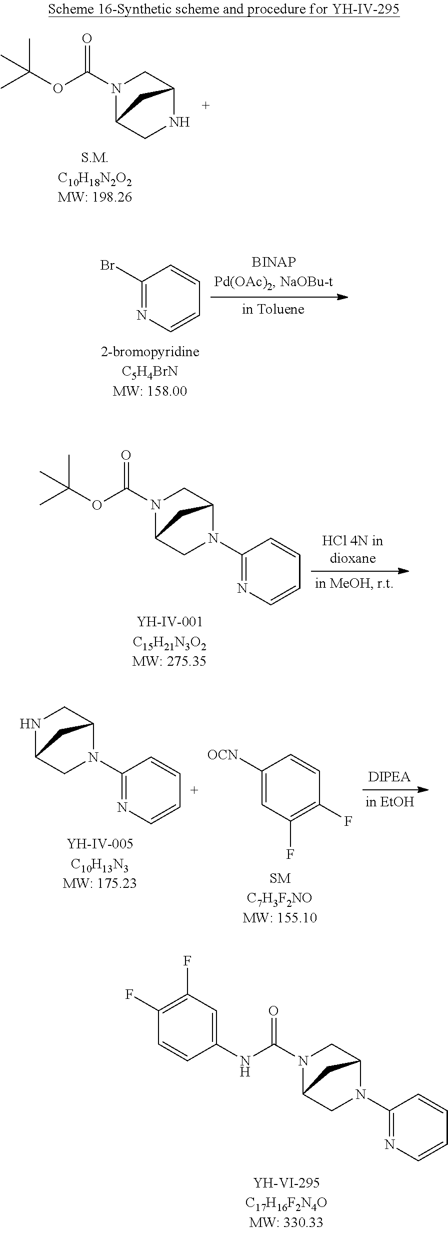

### YH-IV-001: (1R,4R)-tert-butyl 5-(pyridin-2-yl)-2,5-diazabicyclo[2.2.1]heptane-2-carboxylate (C₁₅H₂₁N₃O₂)

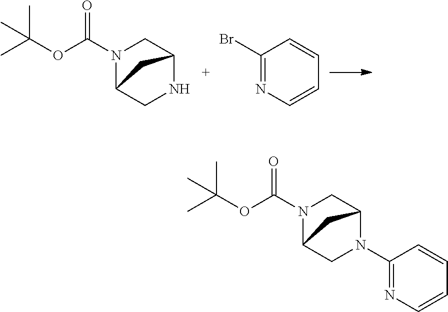 [SMILES: CC(C)(C)OC(=O)N1C[C@H]2C[C@@H]1CN2 ; Brc1ccccn1 ; CC(C)(C)OC(=O)N1C[C@H]2C[C@@H]1CN2c1ccccn1]

To a mixture of (1R,4R)-tert-butyl 2,5-diazabicyclo[2.2.1]heptane-2-carboxylate (1.00 g, 0.005035 mol) in anhydrous toluene (30 mL) was added 2-bromopyridine (1.59 g, 0.01007 mol), NaOBu-t (0.485 g, 0.005035 mol), Pd(OAc)₂ (57 mg, 0.0002522 mol), and (R)-(+)-2,2′-bis(diphenylphosphino)-1,1′-binaphthalene (BINAP, 0.157 g, 0.0002522 mol) at room temperature under the argon atmosphere. The resulting reaction mixture was heated at reflux for 4-5 hours under argon. After the end of the reaction was established by TLC, the reaction was quenched by water, extracted with ethyl acetate. The organic layer was dried with MgSO₄, filtered, and concentrated under vacuum. The product was purified by a silica gel column using hexanes and ethyl acetate (1:1) as eluent to afford 0.96 g (70%) of the desired compound as yellowish solid.

¹HNMR (400 MHz, DMSO-d₆) δ 8.06 (d, J=4.8 Hz, 1H, ArH), 7.51-7.47 (m, 1H, ArH), 6.60-6.57 (m, 1H, ArH), 6.54-6.51 (m, 1H, ArH), 4.77 (d, J=8.8 Hz, 1H, CH), 3.51-3.45 (m, 1H, CH), 3.44-3.30 (m, 2H, CH), 3.26-3.33 (m, 1H, CH), 3.18-3.15 (m, 1H, CH), 1.90-1.87 (m, 2H, CH), 1.39 (s, 6H, 2×CH₃), 1.34 (s, 3H, CH₃).

Mass (ESI, Negative): [M−H]⁻; (ESI, Positive): [M+Na]⁺.

HRMS [C₁₅H₂₂N₃O₂⁺]: calculated 276.1712, found 276.1716 [M+H]⁺. Purity: 99.74% (HPLC).

### YH-IV-005: (1R,4R)-2-(pyridin-2-yl)-2,5-diazabicyclo[2.2.1]heptane (C₁₀H₁₃N₃)

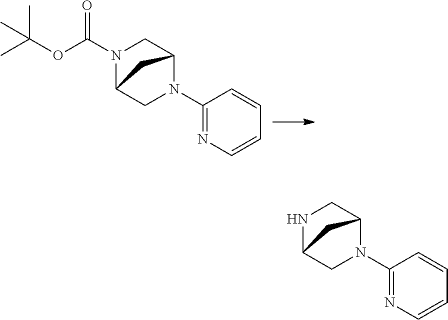 [SMILES: CC(C)(C)OC(=O)N1C[C@H]2C[C@@H]1CN2c1ccccn1 ; c1ccc(N2C[C@H]3C[C@@H]2CN3)nc1]

To a solution of (1R,4R)-tert-butyl 5-(pyridin-2-yl)-2,5-diazabicyclo[2.2.1]heptane-2-carboxylate (0.62 g, 0.0022517 mol) in methanol (10 mL), which was cooled in an ice water bath under an argon atmosphere, was added 2N HCl in Et₂O (10 mL, 0.020265 mol). The resulting reaction mixture was allowed to stir 30 minutes at room temperature under argon. After the end of the reaction was established by TLC, the reaction was concentrated under vacuum to afford 0.60 g as yellowish solid. The product was used for the next step without further purification.

¹HNMR (400 MHz, DMSO-d₆) δ 8.13 (d, J=4.8 Hz, 1H, ArH), 7.48-7.43 (m, 1H, ArH), 6.63-6.58 (m, 1H, ArH), 6.37-6.32 (m, 1H, ArH), 5.03 (d, J=7.8 Hz, 1H, CH), 4.54-4.50 (m, 1H, CH), 3.68-3.65 (m, 1H, CH), 3.61-3.49 (m, 2H, CH), 3.44-3.38 (m, 1H, CH), 2.12-1.91 (m, 2H, CH). Mass (ESI, Negative): [M−H]⁻; (ESI, Positive): 176.06 [M+H]⁺; 198.09 [M+Na]⁺.

### YH-IV-295: (1R,4R)-N-(3,4-difluorophenyl)-5-(pyridin-2-yl)-2,5-diazabicyclo[2.2.1]heptane-2-carboxamide (C17H16F2N4O)

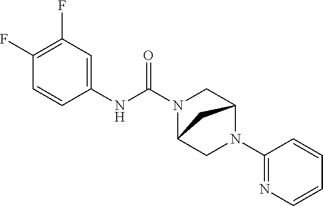 [SMILES: O=C(Nc1ccc(F)c(F)c1)N1C[C@H]2C[C@@H]1CN2c1ccccn1]

To a solution of (1R,4R)-2-(pyridin-2-yl)-2,5-diazabicyclo[2.2.1]heptane (0.172 g, 0.0009791 mol) in anhydrous DCM (10 mL) was added Et3N (0.495 g, 0.004895 mol) and 1,2-difluoro-4-isocyanatobenzene (0.304 g, 0.001958 mol), which was cooled in an ice bath under the argon atmosphere. The resulting reaction mixture was stirred and was homogenous at room temperature under argon for 6-8 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and methanol (19:1 to 9:1) as eluent to afford 0.20 g (61.9%) of the desired compound as white foam.

¹HNMR (400 MHz, DMSO₆) δ 8.47 (s, 1H, NH), 8.08-8.06 (m, 1H, ArH), 7.68-7.62 (m, 1H, ArH), 7.52-7.48 (m, 1H, ArH), 7.31-7.20 (m, 2H, ArH), 6.60-6.53 (m, 2H, ArH), 4.87 (s, 1H, CH), 4.75 (s, 1H, CH), 3.54-3.48 (m, 2H, CH), 3.38-3.30 (m, 2H, CH), 1.99-1.93 (m, 2H, CH).

Mass (ESI, Negative): [M−H]⁻; (ESI, Positive): [M+H]⁺.

HRMS [C₁₇H₁₇F₂N₄O⁺]: calculated 331.1370, found 331.1364. Purity: 96.80% (HPLC).

HRMS [C₁₇H₁₅F₂N₄O⁻]: calculated 329.1214, found. Purity: 96.80% (HPLC).

### Example 18

### Synthesis of YH-IV-299

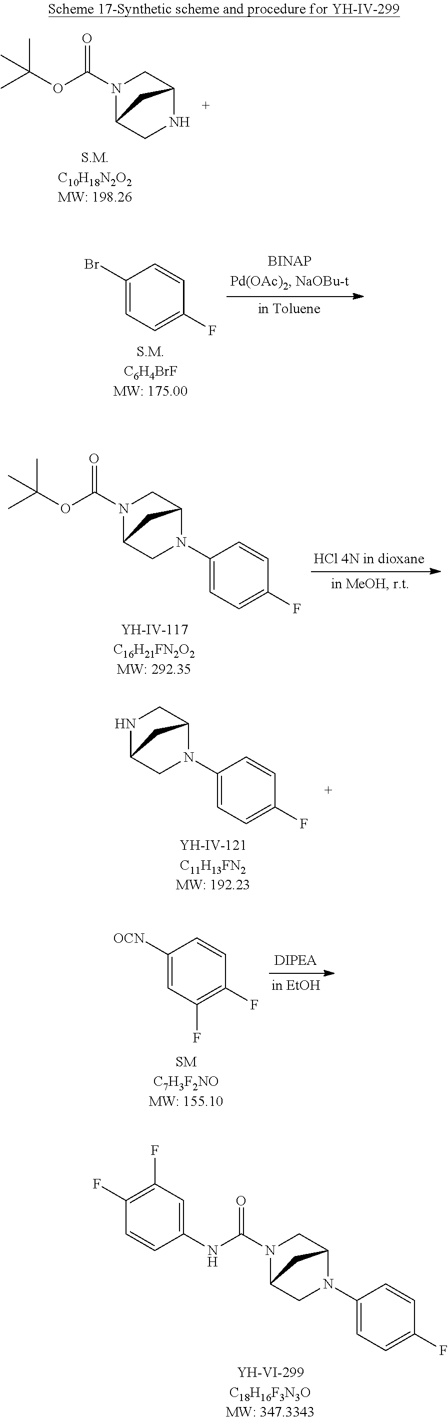

### YH-IV-117: (1R,4R)-tert-butyl 5-(4-fluorophenyl)-2,5-diazabicyclo[2.2.1]heptane-2-carboxylate (C16H21FN2O2)

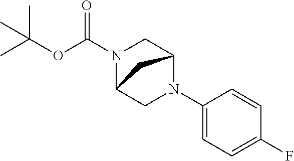 [SMILES: CC(C)(C)OC(=O)N1C[C@H]2C[C@@H]1CN2c1ccc(F)cc1]

To a mixture of (1R,4R)-tert-butyl 2,5-diazabicyclo[2.2.1]heptane-2-carboxylate (2.00 g, 0.010088 mol) in anhydrous toluene (50 mL) was added 1-bromo-4-fluorobenzene (3.53 g, 0.020176 mol), KOBu-t (1.36 g, 0.012105 mol), Pd(OAc)₂ (0.113 g, 0.005044 mol), and (R)-(+)-2,2′-bis(diphenylphosphino)-1,1′-binaphthalene (BINAP, 0.314 g, 0.005044 mol) at room temperature under the argon atmosphere. The resulting reaction mixture was heated at reflux for 12 hours under argon. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (19:1) as eluent to afford 1.00 g (33.9%) of the desired compound as white solid.

¹HNMR (400 MHz, dmso-d₆) δ 7.02-6.97 (m, 2H, ArH), 6.61-6.56 (m, 2H, ArH), 4.46-4.37 (m, 2H, CH), 3.55-3.50 (m, 1H, CH), 3.31-3.24 (m, 1H, CH), 3.20-3.16 (m, 1H, CH), 2.93-2.88 (m, 1H, CH), 1.91-1.84 (m, 2H, CH), 1.39 (s, 6H, 2×CH₃), 1.32 (s, 3H, CH₃).

Mass (ESI, Negative): [M−H]⁻; (ESI, Positive): [M+Na]⁺.

HRMS [C₁₆H₂₂FN₂O₂⁺]: calculated 293.1665, found [M+H]⁺. Purity: 96.93% (HPLC).

### YH-IV-121: (1R,4R)-2-(4-fluorophenyl)-2,5-diazabicyclo[2.2.1]heptane (C11H13FN2)

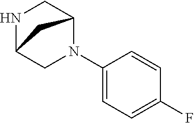 [SMILES: Fc1ccc(N2C[C@H]3C[C@@H]2CN3)cc1]

To a solution of (1R,4R)-tert-butyl 5-(4-fluorophenyl)-2,5-diazabicyclo[2.2.1]heptane-2-carboxylate (0.48 g, 0.001642 mol) in methanol (10 mL), which was cooled in an ice water bath under an argon atmosphere, was added 4N HCl in dioxane (3.70 mL, 0.014777 mol). The resulting reaction mixture was allowed to stir 2-3 hours at room temperature under argon. After the end of the reaction was established by TLC, the reaction was concentrated under vacuum to afford 0.40 g as yellowish solid. The product was used for the next step without further purification.

¹HNMR (400 MHz, dmso-d₆) δ

HRMS [C₁₆H₂₂FN₂O₂⁺]: calculated, found [M+H]⁺. Purity: % (HPLC).

### YH-IV-299: (1R,4R)-N-(3,4-difluorophenyl)-5-(4-fluorophenyl)-2,5-diazabicyclo[2.2.1]heptane-2-carboxamide (C18H16F3N3O)

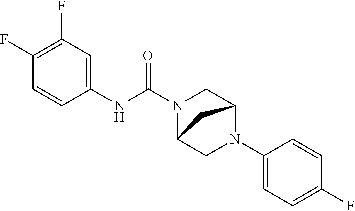 [SMILES: O=C(Nc1ccc(F)c(F)c1)N1C[C@H]2C[C@@H]1CN2c1ccc(F)cc1]

To a solution of (1R,4R)-2-(4-fluorophenyl)-2,5-diazabicyclo[2.2.1]heptane (0.188 g, 0.0009791 mol) in anhydrous DCM (10 mL) was added Et3N (0.495 g, 0.004895 mol) and 1,2-difluoro-4-isocyanatobenzene (0.304 g, 0.001958 mol), which was cooled in an ice bath under the argon atmosphere. The resulting reaction mixture was stirred and was homogenous at room temperature under argon for 6-8 hours. After the end of the reaction was established by TLC, the reaction was quenched by water, extracted with ethyl acetate. The organic layer was dried with MgSO₄, filtered, and concentrated under vacuum. The product was purified by a silica gel column using DCM and ethyl acetate (19:1) as eluent to afford 0.16 g (47.2%) of the desired compound as yellowish foam.

¹HNMR (400 MHz, DMSO₆) δ 8.46 (s, 1H, NH), 7.67-7.61 (m, 1H, ArH), 7.31-7.21 (m, 2H, ArH), 7.04-6.99 (m, 2H, ArH), 6.63-6.61 (m, 2H, ArH), 4.72 (s, 1H, CH), 4.57 (s, 1H, CH), 3.58-3.56 (m, 1H, CH), 3.47-3.37 (m, 2H, CH), 3.02-2.99 (m, 1H, CH), 1.99-1.92 (m, 2H, CH).

Mass (ESI, Negative): [M−H]⁻; (ESI, Positive): [M+H]⁺.

HRMS [C₁₈H₁₇F₃N₃O⁺]: calculated 348.1324, found 348.1319. Purity: 95.78% (HPLC).

HRMS [C₁₈H₁₈F₃N₃O⁻]: calculated 346.1167, found. Purity: 95.78% (HPLC).

### Example 19

### Ability of Bromodomain Inhibitors to Enhance TMZ-Induced GBM Cell Death

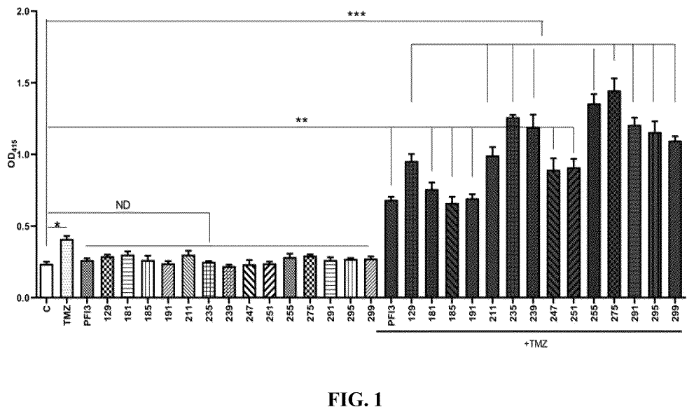

It has been previously shown that PFI-3 enhanced TMZ-induced cell death of GBM cells.^{2, 14} A series of bromodomain inhibitors was developed and synthesized. The biological activity of bromodomain inhibitors was assessed for the ability to enhance TMZ-induced cell death. In brief, MT330 cells were treated with 10 mM of bromodomain inhibitors alone, or in combination with 200 mM of TMZ for 72 hrs, and GBM cell death was determined by a commercial ELISA. As shown in FIG. 1, treatment of MT330 GBM cells with PFI-3 or the bromodomain inhibitors alone did not induce cell death. However, while treatment with TMZ induced low levels of MT330 cell death, PFI-3 treatment markedly increased the sensitivity of GBM cells to TMZ-induced cell death. Importantly, we found that treatment with many of our newly synthesized bromodomain inhibitors (YH-IV-129, YH-IV-211, YH-IV-235, YH-IV-255, and YH-IV-275) had better efficacy than PFI-3 in increasing the cell death-inducing activity of TMZ in GBM cells. In contrast, YH-IV-191 appeared to be biologically inactive as it did not potentiate the effect of TMZ at all.

### Example 20

### Ability of Bromodomain Inhibitors to Enhance the Antiproliferative Action of TMZ on GBM Cells

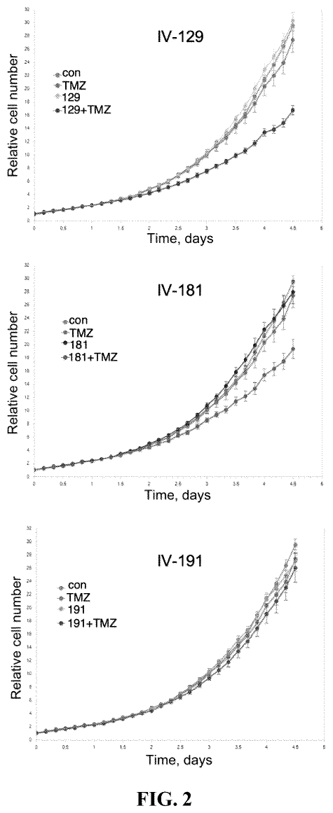

To further characterize the biological activity of selected bromodomain inhibitors, YH-IV-129, YH-IV-181 and YH-IV-191, IncuCyte live cell imaging proliferation assays were performed with the highly TMZ-resistant T98G GBM cell line. Treatment with TMZ or bromodomain inhibitors alone had no inhibitory effect on T98G cell proliferation. In contrast, treatment with YH-IV-129 or YH-IV-181 potently sensitized T98G GBM cells to the antiproliferative action of TMZ (FIG. 2). In marked contrast, treatment with the bromodomain inhibitor YH-IV-191 did not have any effect alone or in combination with TMZ on T98G proliferation.

### Example 21

### Binding of Bromodomain Inhibitors to the Bromodomain of BRG1 and BRM

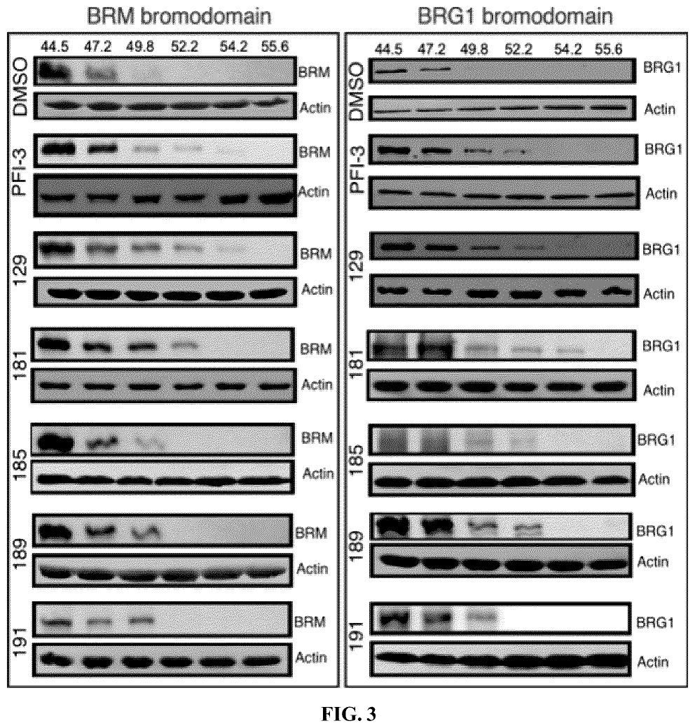

To determine the binding of the bromodomain inhibitors to the BRG1 and BRM bromodomains, a cellular thermal shift assay (CETSA) was developed, which measures the thermostability of the PFI-3 or these analogs in a complex with these bromodomains.^{11, 15} In brief, MT330 cells expressing the BRG1 or BRM bromodomain construct were treated with each bromodomain inhibitors for 2 hr. After heating over a temperature range from 44.5° C. to 55.6° C. for 5 min, MT330 cells were lysed and then immunoblotted for BRG1 or BRM. As shown in FIG. 3, while YH-IV-189 and YH-IV-185 only moderately increased the thermostability of the BRG1 and BRM BRDs, YH-IV-129 and YH-IV-181 significantly increased the thermostability of the BRG1 and BRM BRD expressed in MT330 cells, with the protein band being undetectable at 54.20° C. in bromodomain inhibitors treated cells as compared to the band disappearing at 49.8° C. in the vehicle control. In contrast, YH-IV-191 did not stabilize complex formation with the BRG1 or BRM bromodomain, consistent with this compound not altering the antiproliferative or cell death inducing activity of TMZ.

### Example 22

### Bromodomain Inhibitors Sensitize TMZ-Resistant GBM Cells to TMZ In Vitro

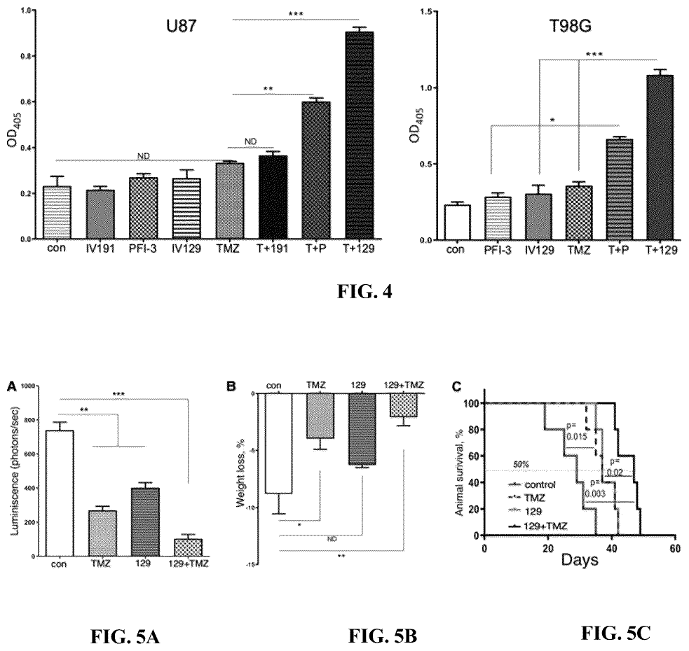

To further test the bioactivity of bromodomain inhibitors, T98G and U87 GBM cell lines were used that are highly resistant to the death inducing activity of TMZ. As further evidence of increased activity, YH-IV-129 was found to be much more potent than PFI-3 in sensitizing U87 and T98G GBM cells in vitro to the cell-death inducing effects of TMZ (FIG. 4). Because of their high TMZ resistance, these cells may serve as cell models for TMZ resistant GBM cancer stem-like cells. In contrast, another BRI, YH-IV-191, was found to be inactive as a chemosensitizer in both TMZ-resistant cell lines, thus demonstrating the specificity of this effect.

### Example 23

### YH-IV-129 Sensitizes GBM to TMZ In Vivo

As YH-IV-129 had markedly enhanced TMZ sensitivity of GBM cells in vitro, we assessed the biological activity of this BRI in an orthotopic GBM model. Luciferase-expressing MT330 cells were injected into the brains of immunocompromised NSG mice. Once tumors were identified by bioluminescence imaging (BLI) at ˜10 days after tumor cell injection, mice were subjected to intraperitoneal injection of TMZ or YH-IV-129 alone, or in combination. At 20 days after initiation of treatment, live animal imaging was performed on an IVIS Lumina instrument. As shown in accompanying FIG. 5, mice treated with a combination of TMZ and YH-IV-129 showed much smaller tumors as evidenced by bioluminescence (BLI) than mice treated with either agent alone. Consistent with the smaller tumor burden in mice treated with a combination of TMZ and YH-IV-129, the weight loss was much lower in mice treated both agents together than in either agent alone (FIG. 5B). The mean survival of control mice was 27 days after cell injection (FIG. 5C). Treatment of TMZ and YH-IV-129 in combination markedly enhanced animal survival (48 days) as compared to either agent alone (35 days). At the drug doses used, YH-IV-129 had no adverse effects on the health, behavior or weight of mice that were not injected with tumor cells, consistent with the absence of “off-target” effects of PFI-3⁸. Based on bioinformatic analysis, YH-IV-129 is expected to have relatively low toxicity, good intestinal absorption, and high blood-brain penetrance, which indicates YH-IV-129 is an excellent lead compound. This is compelling preliminary evidence that bromodomain inhibitors (YH-IV-129) have potent anticancer activity in intracranial GBM xenografts when combined with standard-of-care TMZ chemotherapy and crosses the blood-brain barrier.

### Example 24

### Identification of a BRG1-Selective BRI

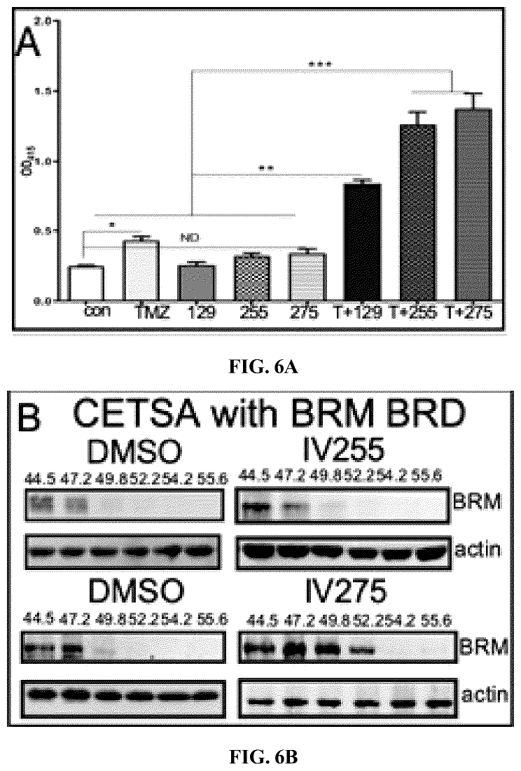

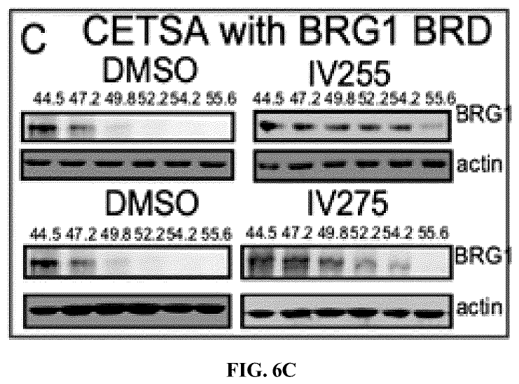

Two new structurally similar bromodomain inhibitors are somewhat more bioactive than YH-IV-129, YH-IV-255 and YH-IV-275 (see FIG. 1). As shown in FIG. 6A, they similarly increase the TMZ sensitivity of GBM cells. However, although YH-IV-275 binds well to the BRM BRD, YH-IV-255 does not bind (FIG. 6B). Both bind equivalently to the BRG1 BRD (FIG. 6C). Thus, the first BRD inhibitor that is BRG1-specific was identified. This compound can be optimized further for bioactivity and the ability to cross the BBB. Based on bioinformatic analysis, YH-IV-255 is predicted to readily cross the BBB, have excellent intestinal absorption, and minimal off-target effects.

### Example 25

### The Bioactivity of Bromodomain Inhibitor YH-IV-255 Requires the Presence of BRG1

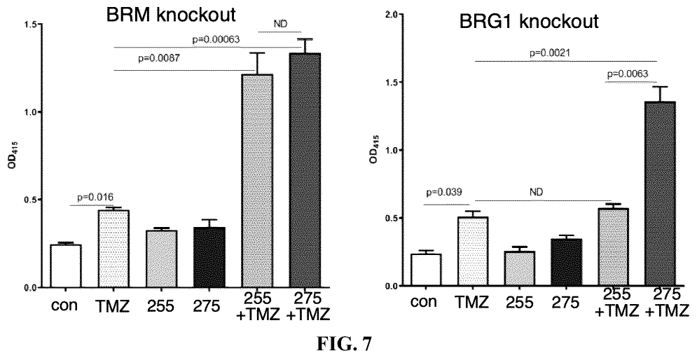

To further characterize the specificity of YH-IV-255 for the BRG1 bromodomain, GBM cell lines were generated with either BRG1 or BRM knocked out by CRISPR/Cas9 gene editing.¹⁴ These cells were isolated by puromycin selection and the knockout of the either subunit was validated by immunoblotting. As shown in FIG. 7, while YH-IV-255 and YH-IV-275 have very similar activity in enhancing the cell death inducing activity in BRM knockout GBM cells, YH-IV-255 has no such activity in BRG1 knockout GBM cells. This finding provides another independent form of evidence that YH-IV-255 is a selective BRG1 bromodomain inhibitor. In contrast, YH-IV-275, which only differs by a single chemical substitution from YH-IV-255, is an inhibitor of both BRG1 and BRM domains.

### Example 26

### Bromodomain Inhibitors Sensitize GBM Cells to Bleomycin, a Mimic of Ionizing Radiation

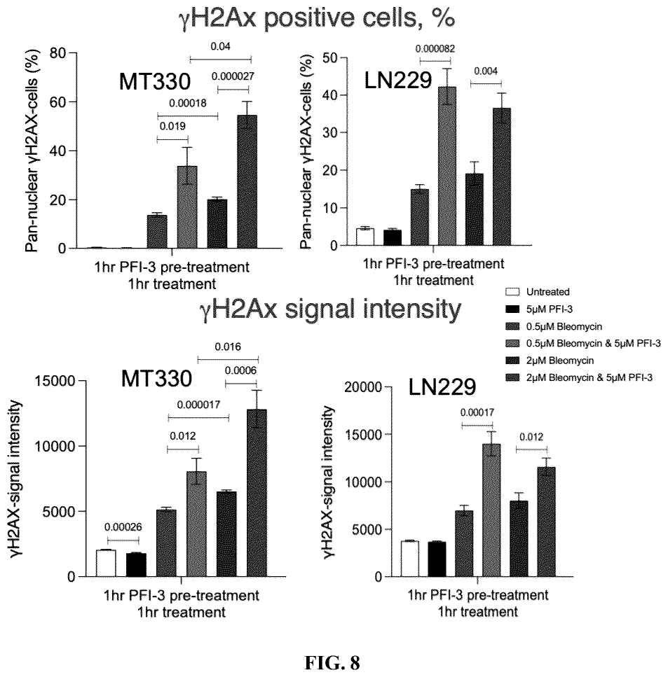

Bleomycin is a chemotherapeutic drug that is used to treat various cancers. In addition, bleomycin induces DNA damage in a manner like that of ionizing radiation, and hence is used as an in vitro mimic of ionizing radiation. Thus, the ability of bleomycin to induce DNA damage as evidence by immunostaining of MT330 cells and LN229 with γH2Ax was tested. Quantification of cell staining was determined on the Lionheart FX imaging station. As shown in FIG. 8, bleomycin induced a dose dependent increase of γH2Ax staining in the number of γH2Ax positive cells and the amount of overall γH2Ax staining. Whereas PFI-3 alone had an no effect on γH2Ax staining, PFI-3 pretreatment of cells resulted in an increased number of both γH2Ax positive cells and signal intensity of staining upon bleomycin treatment, showing that PFI-3 treatment also sensitized GBM cells to DNA damaged induced by a mimic of ionizing radiation.

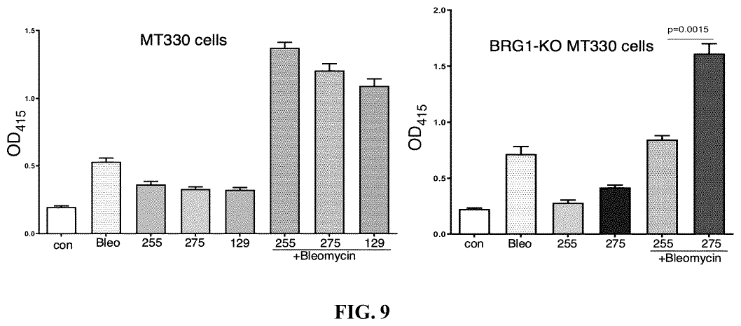

Based on this evidence that PFI-3 enhanced the effects of bleomycin, the ability of bromodomain inhibitors to enhance the cell death inducing activity of bleomycin was examined. As shown in FIG. 9 and consistent with prior work, none of the bromodomain inhibitors (YH-IV-129, YH-IV-255 and YH-IV-275) alone had any death inducing activity in GBM cells. Whereas low dose of bleomycin (0.01 mM) induced low levels of cell death in MT330 cells, bleomycin in combination with bromodomain inhibitors had a marked effect on MT330 cell death. Furthermore, consistent with our results with TMZ induced cell death, the marked effect of YH-IV-255 with TMZ was ablated in BRG1 knockout MT330 cells. This data provides additional evidence for YH-IV-255 being a selective inhibitor the BRG-1 bromodomain, which potentiates the effects of TMZ as well as ionizing radiation.

### Example 27

### Bromodomain Inhibitors Sensitize GBM to TMZ In Vitro and In Vivo

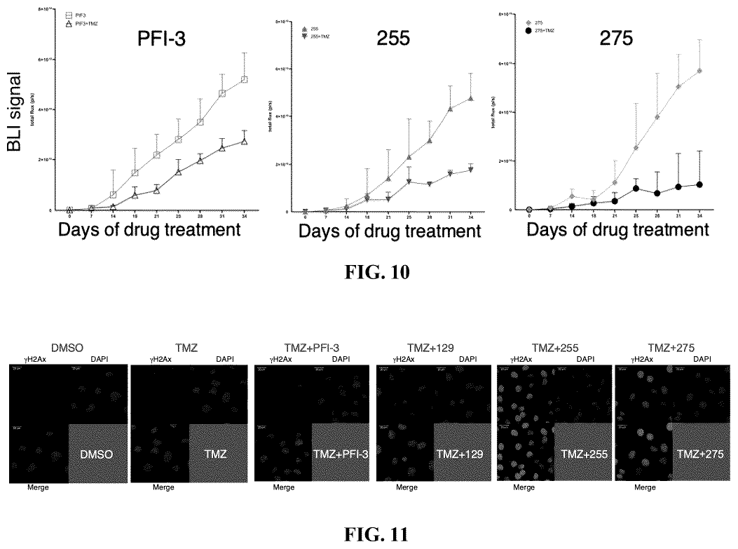

As bromodomain inhibitors YH-IV-255 and YH-IV-275 markedly enhanced TMZ sensitivity of GBM cells in vitro as compared to PFI-3, the biological activity of these analogs was assessed in an orthotopic GBM model. Luciferase-expressing MT330 cells were injected into the brains of immunocompromised NSG mice. Once tumors were identified by bioluminescence imaging (BLI) at ˜10 days after tumor cell injection, mice were subjected to intraperitoneal injection of TMZ or YH-IV-255 or YH-IV-275 alone, or in combination. Treatment of TMZ and YH-IV-255 or YH-IV-275 in combination decreased the bioluminescent signal in the brain after 3-4 weeks after treatment as compared to either agent alone (FIG. 10), indicating that at the doses used these bromodomain inhibitors in combination with TMZ markedly slowed brain tumor growth. At the drug doses used, the bromodomain inhibitors used had no adverse effects on the health, behavior or weight of mice that were not injected with tumor cells, consistent with the absence of “off-target” effects of PFI-3.⁶⁻⁷ Based on bioinformatic analysis, YH-IV-255 and YH-IV-275 are expected to have relatively low toxicity, good intestinal absorption, and high blood-brain penetrance, which indicates that they are excellent therapeutic compounds. This is compelling preliminary evidence that these two new compounds have potent anticancer activity in intracranial GBM xenografts when combined with standard-of-care TMZ chemotherapy and that these drugs cross the blood-brain barrier.

### Example 28

### Bromodomain Inhibitors Enhance DNA Damage Induced by TMZ

As TMZ is DNA damaging agent, we tested the ability of various bromodomain inhibitors to enhance TMZ-induced DNA damage as evidence by immunostaining of GBM cells with γH2Ax and examination of the staining on a confocal microscope. γH2AX is the phosphorylated version of the histone variant H₂Ax and is a highly specific and sensitive marker for monitoring DNA damage initiation and resolution. As shown in FIG. 11, TMZ induced a clear and detectable increase of γH2Ax staining in LN229 GBM cells. Whereas treatment with bromodomain inhibitors alone had no detectable effect on γH2Ax staining, pretreatment of cells with PFI-3, YH-IV-255 or YH-IV-275 resulted in a marked increase in γH2Ax staining. Most interestingly, YH-IV-255 and YH-IV-275 potentiated the amount γH2Ax staining of showing that BRI treatment sensitized GBM cells to DNA damage. Similar results were obtained with MT330 GBM cells.

The various features and processes described above may be used independently of one another or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Whereas certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

To the extent that the appended claims have been drafted without multiple dependencies, this has been done only to accommodate formal requirements in jurisdictions which do not allow such multiple dependencies. It should be noted that all possible combinations of features which would be implied by rendering the claims multiply dependent are explicitly envisaged and should be considered part of the invention.

### REFERENCES

Several patents and publications are cited above in order to more fully describe and disclose the invention and the state of the art to which the invention pertains. Full citations for these references are provided below. All references cited herein are incorporated herein by reference in their entirety. To the extent publications and patents or patent applications incorporated by reference contradict the disclosure contained in the specification, the specification is intended to supersede and/or take precedence over any such contradictory material.
ADDIN EN.REFLIST (1) Surawicz, T. S.; Davis, F.; Freels, S.; Laws, E. R., Jr.; Menck, H. R. Brain tumor survival: results from the National Cancer Data Base. J Neurooncol 1998, 40 (2), 151-160. DOI: 10.1023/a:1006091608586.
(2) Ganguly, D.; Sims, M.; Cai, C.; Fan, M.; Pfeffer, L. M. Chromatin Remodeling Factor BRG1 Regulates Stemness and Chemosensitivity of Glioma Initiating Cells. Stem Cells 2018, 36 (12), 1804-1815. DOI: 10.1002/stem.2909.
(3) Tamkun, J. W.; Deuring, R.; Scott, M. P.; Kissinger, M.; Pattatucci, A. M.; Kaufman, T. C.; Kennison, J. A. brahma: a regulator of Drosophila homeotic genes structurally related to the yeast transcriptional activator SNF2/SWI2. Cell 1992, 68 (3), 561-572. DOI: 10.1016/0092-8674(92)90191-e.
(4) Haynes, S. R.; Dollard, C.; Winston, F.; Beck, S.; Trowsdale, J.; Dawid, I. B. The bromodomain: a conserved sequence found in human, Drosophila and yeast proteins. Nucleic Acids Res 1992, 20 (10), 2603. DOI: 10.1093/nar/20.10.2603.
(5) Filippakopoulos, P.; Picaud, S.; Mangos, M.; Keates, T.; Lambert, J. P.; Barsyte-Lovejoy, D.; Felletar, I.; Volkmer, R.; Muller, S.; Pawson, T.; et al. Histone recognition and large-scale structural analysis of the human bromodomain family. Cell 2012, 149 (1), 214-231. DOI: 10.1016/j.cell.2012.02.013.
(6) Ntranos, A.; Casaccia, P. Bromodomains: Translating the words of lysine acetylation into myelin injury and repair. Neurosci Lett 2016, 625, 4-10. DOI: 10.1016/j.neulet.2015.10.015.
(7) Medina, P. P.; Carretero, J.; Fraga, M. F.; Esteller, M.; Sidransky, D.; Sanchez-Cespedes, M. Genetic and epigenetic screening for gene alterations of the chromatin-remodeling factor, SMARCA4/BRG1, in lung tumors. Genes Chromosomes Cancer 2004, 41 (2), 170-177. DOI: 10.1002/gcc.20068.
(8) Gerstenberger, B. S.; Trzupek, J. D.; Tallant, C.; Fedorov, O.; Filippakopoulos, P.; Brennan, P. E.; Fedele, V.; Martin, S.; Picaud, S.; Rogers, C.; et al. Identification of a Chemical Probe for Family VIII Bromodomains through Optimization of a Fragment Hit. Journal of medicinal chemistry 2016, 59 (10), 4800-4811. DOI: 10.1021/acs.jmedchem.6b00012.
(9) Fedorov, O.; Castex, J.; Tallant, C.; Owen, D. R.; Martin, S.; Aldeghi, M.; Monteiro, O.; Filippakopoulos, P.; Picaud, S.; Trzupek, J. D.; et al. Selective targeting of the BRG/PB1 bromodomains impairs embryonic and trophoblast stem cell maintenance. Sci Adv 2015, 1 (10), e1500723. DOI: 10.1126/sciadv.1500723.
(10) Philpott, M.; Yang, J.; Tumber, T.; Fedorov, O.; Uttarkar, S.; Filippakopoulos, P.; Picaud, S.; Keates, T.; Felletar, I.; Ciulli, A.; et al. Bromodomain-peptide displacement assays for interactome mapping and inhibitor discovery. Mol Biosyst 2011, 7 (10), 2899-2908. DOI: 10.1039/c1mb05099k. Ciceri, P.; Muller, S.; O'Mahony, A.; Fedorov, O.; Filippakopoulos, P.; Hunt, J. P.; Lasater, E. A.; Pallares, G.; Picaud, S.; Wells, C.; et al. Dual kinase-bromodomain inhibitors for rationally designed polypharmacology. Nat Chem Biol 2014, 10 (4), 305-312. DOI: 10.1038/nchembio.1471.
(11) He, Y.; Yang, C.; Wang, Y.; Sacher, J. R.; Sims, M. M.; Pfeffer, L. M.; Miller, D. D. Novel structural-related analogs of PFI-3 (SRAPs) that target the BRG1 catalytic subunit of the SWI/SNF complex increase the activity of temozolomide in glioblastoma cells. Bioorganic & medicinal chemistry 2022, 53, 116533. DOI: 10.1016/j.bmc.2021.116533.
(12) Yang, C. W., Y.; Sims, M. M.; He, Y.; Miller, D. D. and Pfeffer, L. M. Targeting the Bromodomain of BRG-1/BRM Subunit of the SWI/SNF Complex Increases the Anticancer Activity of Temozolomide in Glioblastoma. Pharmaceuticals 2021, 14 (9), 904. DOI: 10.3390/ph14090904.
(13) Wang, Y.; Yang, C. H.; Schultz, A. P.; Sims, M. M.; Miller, D. D.; Pfeffer, L. M. Brahma-Related Gene-1 (BRG1) promotes the malignant phenotype of glioblastoma cells. J. Cell. Mol. Med. 2021, 25 (6), 2956-2966. DOI: 10.1111/jcmm.16330.
(14) Yang, C.; Wang, Y.; Sims, M. M.; He, Y.; Miller, D. D.; Pfeffer, L. M. Targeting the Bromodomain of BRG-1/BRM Subunit of the SWI/SNF Complex Increases the Anticancer Activity of Temozolomide in Glioblastoma. Pharmaceuticals (Basel) 2021, 14 (9). DOI: 10.3390/ph14090904.
(15) Yang, C.; Wang, Y.; Sims, M. M.; He, Y.; Miller, D. D.; Pfeffer, L. M. Targeting the Bromodomain of BRG-1/BRM Subunit of the SWI/SNF Complex Increases the Anticancer Activity of Temozolomide in Glioblastoma. Pharmaceuticals 2021, 14 (9), 904.

What is claimed is:

1. A compound of Formula (I): 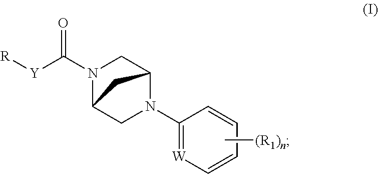
a stereoisomers thereof, a prodrug thereof, or a pharmaceutically acceptable salts thereof wherein:
R represents alkyl, cycloalkyl, alkenyl, cycloalkyl, alkynyl, phenyl, aryl, heteroaryl or arylalkyl moiety having at most 12 carbon atoms, these moieties being optionally substituted by one or more substituents chosen from halogen atoms and the following functional groups:
optionally esterified. etherified or protected hydroxy, alkoxy, hydroxyalkyl, alkenyloxy, alkynyloxy, trifluoromethyl, mercapto, halogens, cyano, nitro, acyl, acyloxy, aryl, free, esterified, amidified or salified carboxy, amino, mono- or dialkylamino, or hydrogen.
R₁=alkyl, alkoxy, hydroxy, esterified, etherified or protected hydroxy, amino, mono- or dialkylamino, halogens (F, Cl, Br, I), acyl, acyloxy, cyano, trifluoromethyl, methoxy, nitro, esterified, amidified or salified carboxy, and hydrogen;
Y=bond, O, NH, CH₂;
W═CH, N;
n is an integer from 0 to 4.

2. The compound of claim 1, selected from the structures: 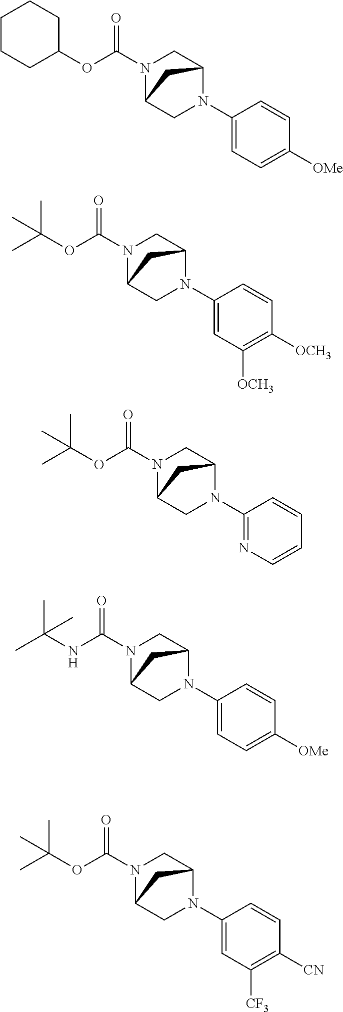 [SMILES: COc1ccc(N2C[C@H]3C[C@@H]2CN3C(=O)OC2CCCCC2)cc1 ; COc1ccc(N2C[C@H]3C[C@@H]2CN3C(=O)OC(C)(C)C)cc1OC ; CC(C)(C)OC(=O)N1C[C@H]2C[C@@H]1CN2c1ccccn1 ; COc1ccc(N2C[C@H]3C[C@@H]2CN3C(=O)NC(C)(C)C)cc1 ; CC(C)(C)OC(=O)N1C[C@H]2C[C@@H]1CN2c1ccc(C#N)c(C(F)(F)F)c1]
or a pharmaceutically acceptable salt thereof.

3. A pharmaceutical composition comprising the compound of claim 1, further comprising at least one pharmaceutically acceptable carrier.

4. A compound having the structure of Formula (II): 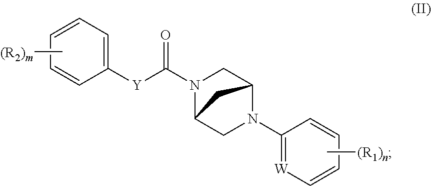
a stereoisomers thereof, a prodrug thereof, or a pharmaceutically acceptable salts thereof wherein R₁=alkyl, alkoxy, hydroxy, esterified, etherified or protected hydroxy, amino, mono- or dialkylamino, halogens (F, Cl, Br, I), acyl, acyloxy, cyano, trifluoromethyl, methoxy, nitro, esterified, amidified or salified carboxy, and hydrogen;
R₂=alkyl, alkoxy, hydroxy, esterified, etherified or protected hydroxy, amino, mono- or dialkylamino, halogens (F, Cl, Br, I), acyl, acyloxy, cyano, trifluoromethyl, methoxy, nitro, esterified, amidified or salified carboxy, and hydrogen;
Y=bond, O, NH, CH₂;
W═CH, N;
m or n is an integer from 0 to 4.

5. A compound of claim 3, selected from the structures: 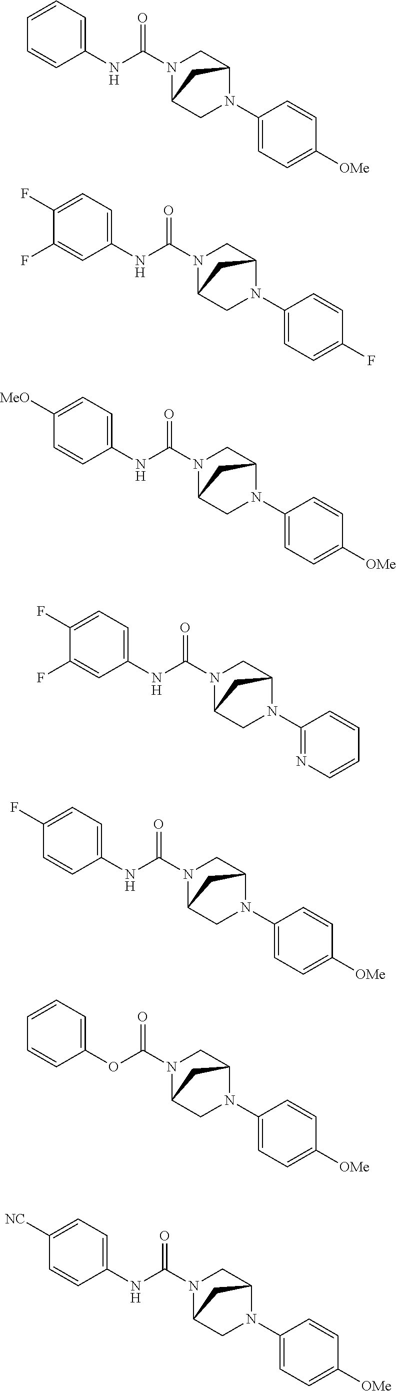 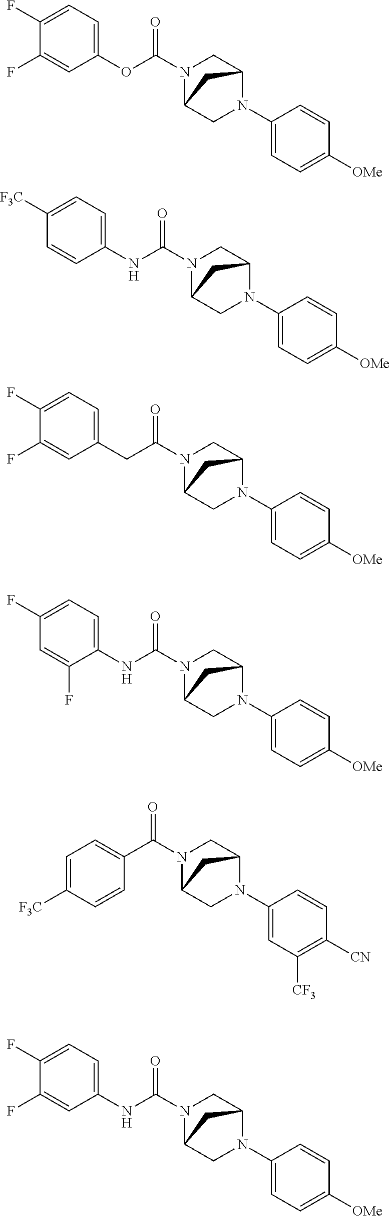 [SMILES: COc1ccc(N2C[C@H]3C[C@@H]2CN3C(=O)Oc2ccc(F)c(F)c2)cc1 ; COc1ccc(N2C[C@H]3C[C@@H]2CN3C(=O)Nc2ccc(C(F)(F)F)cc2)cc1 ; COc1ccc(N2C[C@H]3C[C@@H]2CN3C(=O)Cc2ccc(F)c(F)c2)cc1 ; COc1ccc(N2C[C@H]3C[C@@H]2CN3C(=O)Nc2ccc(F)cc2F)cc1 ; N#Cc1ccc(N2C[C@H]3C[C@@H]2CN3C(=O)c2ccc(C(F)(F)F)cc2)cc1C(F)(F)F ; COc1ccc(N2C[C@H]3C[C@@H]2CN3C(=O)Nc2ccc(F)c(F)c2)cc1]
or a pharmaceutically acceptable salt thereof.

6. A pharmaceutical composition comprising the compound of claim 4, further comprising at least one pharmaceutically acceptable carrier.

7. A method of treating cancer in a subject in need comprising administering a therapeutically effective amount of a compound of Formula (I) to the subject: 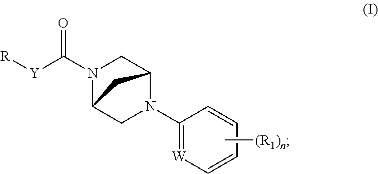
a stereoisomers thereof, a prodrug thereof, or a pharmaceutically acceptable salts thereof wherein:
R represents alkyl, cycloalkyl, alkenyl, cycloalkyl, alkynyl, phenyl, aryl, heteroaryl or arylalkyl moiety having at most 12 carbon atoms, these moieties being optionally substituted by one or more substituents chosen from halogen atoms and the following functional groups:
optionally esterified, etherified or protected hydroxy, alkoxy, hydroxyalkyl, alkenyloxy, alkynyloxy, trifluoromethyl, mercapto, halogens, cyano, nitro, acyl, acyloxy, aryl, free, esterified, amidified or salified carboxy, amino, mono- or dialkylamino, or hydrogen.
R₁=alkyl, alkoxy, hydroxy, esterified, etherified or protected hydroxy, amino, mono- or dialkylamino, halogens (F, Cl, Br, I), acyl, acyloxy, cyano, trifluoromethyl, methoxy, nitro, esterified, amidified or salified carboxy, and hydrogen;
Y=bond, O, NH, CH₂;
W═CH, N;
n is an integer from 0 to 4.

8. The method of claim 7, wherein the cancer is glioblastoma.

9. The method of claim 7, wherein the compound is administered to the subject with a pharmaceutical composition.

10. The method of claim 7, wherein the pharmaceutical composition is administered in combination with one or more DNA alkylating agents.

11. The method of claim 10, wherein said DNA alkylating agent is temozolomide.

12. A method of treating cancer in a subject in need comprising administering a therapeutically effective amount of a compound of Formula (II) to the subject: 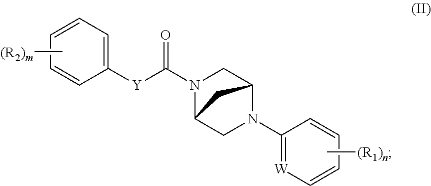
a stereoisomers thereof, a prodrug thereof, or a pharmaceutically acceptable salts thereof wherein:
R represents alkyl, cycloalkyl, alkenyl, cycloalkyl, alkynyl, phenyl, aryl, heteroaryl or arylalkyl moiety having at most 12 carbon atoms, these moieties being optionally substituted by one or more substituents chosen from halogen atoms and the following functional groups: optionally esterified. etherified or protected hydroxy, alkoxy, hydroxyalkyl, alkenyloxy, alkynyloxy, trifluoromethyl, mercapto, halogens, cyano, nitro, acyl, acyloxy, aryl, free, esterified, amidified or salified carboxy, amino, mono- or dialkylamino.
R1=alkyl, alkoxy, hydroxy, esterified, etherified or protected hydroxy, amino, mono- or dialkylamino, halogens (F, Cl, Br, I), acyl, acyloxy, cyano, trifluoromethyl, methoxy, nitro, esterified, amidified or salified carboxy, and hydrogen;
Y=bond, O, NH, CH₂;
W═CH, N;
n is an integer from 0 to 4.

13. The method of claim 12, wherein the cancer is glioblastoma.

14. The method of claim 12, wherein the compound is administered to the subject with a pharmaceutical composition.

15. The method of claim 14, wherein the pharmaceutical composition is administered in combination with one or more DNA alkylating agents.

16. The method of claim 15, wherein said DNA alkylating agent is temozolomide.
